(19) United States
(12) Patent Application Publication
(10) Pub. No.: US 2004/0078219 A1
(43) Pub. Date: Apr. 22, 2004

(54) Healthcare networks with biosensors

(75) Inventors: Rosann Kaylor, Cumming, GA (US); Dennis Everhart, Alpharetta, GA (US); Jeff Lindsay, Appleton, WI (US)

Correspondence Address: STEPHEN E. BONDURA, ESQ., DORITY & MANNING, P.A., P.O. BOX 1449, GREENVILLE, SC, 29602-1449, US

(73) Assignee: KIMBERLY-CLARK WORLDWIDE, INC.

(21) Appl. No.: 10/277,170

(22) Filed: Oct. 21, 2002

(51) Int. Cl. G06F 17/60; A61B 5/00

(52) U.S. Cl. 705/2; 600/300

(57) ABSTRACT

A healthcare network is provided for sharing information concerning the health of a user with at least one outside source, the network including a biosensor associated with the user that generates a biosensor signal containing the information; and a personal data control means including receiving means for receiving the biosensor signal, input means for receiving a privacy input from the user, and output means for generating a response signal based on the biosensor signal and privacy input. The network also includes a data allocation and processing module including means for receiving the response signal, and means for generating and directing an output signal to the at least one outside source, wherein the module is responsive to the response signal, and wherein the availability of the information to the at least one outside source is responsive to the privacy input.

### BACKGROUND

Biosensors have long been an important part of health care in hospitals and some managed care facilities. Recently many technologies have been proposed for biosensors that can be used at home, including disposable or single-use devices. Further, technologies have been proposed that could be incorporated into another item that is worn on or near the body, such as a disposable diaper, incontinence device, sanitary napkin, an article of clothing, and the like. Finally, it has also been proposed to use portable or disposable biosensors equipped with electronic devices that can store or transmit data relevant to the health of a subject.

Biosensors for personal use at home or, more generally, outside of hospitals or medical clinics, offer many opportunities for improved health care.

### SUMMARY OF THE INVENTION

While many parties have proposed health care systems involving transmission of data from biosensors to doctors or other caregivers to improve the care of a patient, such systems have not been integrated with health care systems in ways that provide comprehensive services or benefits to the subject while protecting the privacy of the subject. Personal privacy becomes a particular concern for would-be users of biosensors that transmit data via electronic means (wired or wireless) to a remote location such as a hospital. There are fears that data may misrepresent the patient's health without recourse, given the distance between the patient and whatever body interprets the data. There are fears that the data will be misrouted or intercepted by third parties, allowing confidential information about the health of the patient to be known by others such as an employer or insurer. There are fears that spurious signals from the biosensor may be sent, resulting in improper treatment or unnecessary changes in medication.

Further, the highly personal nature of biosensor information collected in a private setting, such as a home or workplace, raises additional concerns about the need for privacy. What is needed is a system that can electronically integrate biosensors in a healthcare network while preserving the user's privacy and sense of control over information provided by the biosensor, and optionally providing means for a user or a representative of the user to provide annotation or comments about apparent biosensor readings or possible problems therewith.

The present invention relates to an integrated health care system employing biosensors capable of generating signals relating to the health of the user that can be processed and transmitted as needed to various destinations, wherein the user or representative of the user maintains a degree of control over the data transmitted for protection of the user's privacy or other considerations. The invention further relates to particular combinations of sensor technologies and information management systems and/or health management systems for the benefit of the user, including embodiments wherein a degree of personal control over data sharing is maintained for user privacy.

In one embodiment, the present invention relates to a healthcare network for sharing information concerning the health of a user with one or more outside sources, including:

a) a biosensor cooperatively associated with the user that generates a biosensor signal pertaining to the health of the user;

b) a personal data control means including means for receiving the biosensor signal, input means for receiving a privacy input from the user or representative of the user, and output means for generating a response signal based on the biosensor signal and privacy input; and

c) a data allocation and processing module including means for receiving the response signal from the personal data control means and means for directing one or more output signals to the one or more outside sources, responsive to the response signal, wherein the availability to the one or more outside sources of health-related information pertaining to the user is responsive to the privacy input.

The healthcare network can further include treatment means for delivering a medication, nutritional substance, medical therapy, or other physical or medical care to the user, responsive to the output signal to the one or more outside sources.

In another aspect, the present invention relates to a method for sharing information concerning the health of a user with one or more outside sources, including:

a) providing a biosensor cooperatively associated with the body of a user, wherein the biosensor generates a biosensor signal pertaining to the health of the user;

b) providing a reading to the user or a representative of the user indicating a preliminary interpretation of the biosensor signal;

c) receiving a privacy input from the user or a representative of the user through input means;

d) generating a response signal based on the biosensor signal and the privacy input;

e) receiving the response signal at a data allocation and processing module, which in turn generates one or more output signals to the one or more outside sources, responsive to the response signal, wherein the availability to the one or more outside sources of health-related information pertaining to the user is responsive to the privacy input.

An electronic personal data control means can be used in performing steps b, c, and d in the above method. The method can further include providing an adjustment in care to the subject in response to the output signal as directed by at least one of the one or more outside sources.

In one embodiment the subject is monitored with at least one biosensor while at a remote location relative to a hospital or other medical care facility. For example, the subject can be at home, in a managed care facility, at the subject's workplace, outdoors, traveling, in a prison, in a military setting (e.g., in a submarine, tank, or airplane), and the like.

A biosensor signal or a signal derived from a biosensor signal can be transmitted to a private database or databases for review by outside sources such as a physician or nurse, but the transmission of data and optionally the availability of that data to other parties is controlled by the user. As used herein, the “user” of a biosensor refers to either the subject or a representative of the subject, such as a parent, family member, someone with power of attorney, or other authorized party. The subject is generally human but can be another species, such as a pet or farm animal, in which case a human representative (the owner, for example) would typically provide the privacy input.

Typically, the biosensor signal is used to generate an intermediate reading or other signal that can be interpreted by a subject or other caregiver, which can permit the user to decide whether the data or information derived therefrom should be forwarded to or made available to outside sources. Decisions about control and availability of the data can be made and revised repeatedly or can be made only once, if desired.

Means can also be provided to generate an alert signal to the subject, a caregiver, or other party based on abnormal biosensor readings that may indicate a health problem. The alert signal may also automatically initiate a call to emergency personnel or application of a responsive treatment, or may require review of an outside party such as a doctor before the treatment is automatically administered. Software and hardware means may also be provided to distinguish an abnormal reading from a hardware problem, such as a disconnected electrode or improper use of the biosensor. Neural networks and fuzzy logic systems may be incorporated to make this distinction.

Private control of the data generated by a biosensor is achieved via a personal data control means, which can include hardware and software for display and tentative interpretation of the biosensor signal(s), input means for receiving a privacy input from the user, and transmission means to direct the resulting response signal (a signal based on the biosensor signal and a privacy input from the subject) to a device for data allocation and processing, where data control instructions responsive to the privacy input are used to direct one or more output signals to one or more outside parties such as a doctor, insurer, employer, and the like.

The data allocation module can employ tools disclosed in U.S. Pat. No. 5,974,389, “Medical Record Management System and Process with Improved Workflow Features,” issued Oct. 26, 1999 to Clark et al., incorporated herein by reference. The disclosed patient medical record system of Clark et al. includes a number of caregiver computers, and a patient record database with patient data coupled to the caregiver computers selectively providing access to the patient data from one of the caregiver computers responsive to a predetermined set of access rules. As adapted for the present invention, the access rules can be modified responsive to the privacy input from the biosensor user.

The privacy input can include instructions about how data or other information pertaining to or derived from the biosensor signal may or may not be used and with whom the data or subsets of the data may be shared. Alternatively or in addition, the privacy input can include optional comments and other restrictions pertaining to the data. In one embodiment, the privacy input can be determined by user options that the user (either the subject or a representative of the subject) selects prior to measurement, or can include privacy settings entered after reviewing data derived from the biosensor signal.

Means may be provided to automatically override a privacy setting when the biosensor may indicate a life-threatening condition or other condition requiring emergency response, or such means may be part of an initial setting approved by the user that can override subsequent selections.

The input means for entering a privacy input can include any suitable data entry means, such as a keyboard connected to a computer, a voice recognition device, a hardware setting such as a button or dial, a toggle switch, and the like, and can be provided by software settings, as in a file specifying user options. Symbolic entry using penstrokes or other interpretable motions can also be used.

Data allocation and processing can be performed with hardware and/or software that is part of the personal data control means, or can occur on a separate server or other means. The output signal forwarded by the data allocation and processing function may then be used by professional staff or other competent parties to adjust medications or other primary care functions provided to the subject, to recommend that the subject be given further testing or examination, to call for emergency assistance, to authorize payment by an insurer or other party, to verify other claims made by the user, or for other purposes typically related to the well-being of the subject.

To facilitate data transfer between the biosensor, the data allocation and processing module, and outside sources, any or all of these elements can follow communication standards such as those Connectivity Industry Consortium (CIC) as described by Alan Reder, “Regulating the Point of Care: The IVD Connectivity Industry Consortium,” Medical Device & Diagnostic Industry, April 2001, available now at www.devicelink.com/mddi/archive/01/04/001.html.

For example, standards can be applied for cabled (RS-232) and wireless (infrared) connectivity. Protocols such as IEEE 1451.2 identify transducer electronic data sheets to enable various sensors to connect to a single node, or pick-and-place technologies can be used to produced integrated systems. Wireless systems can employ systems from the Bluetooth™ Special Interest Group, employing radio-transmitting microchips to allow communication between devices. Access to the data allocation and processing module can follow an industry standard for connecting to networked databases and servers. A common access means can be used that is also suited for existing IEEE 1073 medical information bus (MIB) devices, as well as by all personal digital-assistant devices, cell phones, and laptop computers that have infrared data association (IrDA) ports.

A plurality of subjects at one or more locations may be monitored with the healthcare network of the present invention, each being monitored by one or more biosensors and each optionally having some degree of control over the use of data generated by or derived from biosensors or associated equipment.

The “outside sources” in the healthcare network can include any of the following: doctors, nurses, dentists, and other medical staff at a hospital or other care facility, medical and dental insurers, life insurance agencies, pharmacists and any other providers of medications or health care devices or therapies, public officers such as police or probation officers, employers and associated personnel (e.g., airline supervisors monitoring a pilot or military staff monitoring biosensor signals from soldiers), and so forth. Doctors can include family doctors, pediatricians, surgeons, nephrologists, hematologists, oncologists, gynecologists, dermatologists, and specialists in any other branch of medicine. The associated databases or information management systems for each of the above-mentioned entities can also be included in the healthcare network. In one embodiment, data is transferred to the laboratory information system (LIS) of a hospital or other medical facility. An outside source can include enterprise information systems, such as a clinical data repository (CDR) and electronic medical record (EMR) systems featuring electronic data interchange (EDI) systems. The EDI interface can be built on a standard HL7 messaging scheme.

The biosensor signal can also be received and processed with the hospital network infrastructure described in U.S. pat. appl. Ser. No. 60/135,057, filed May 20, 1999, incorporated herein by reference, and published Nov. 30, 2000 as WO 00/72180 by R. D. Bucholz. This application describes a medical networking infrastructure intended for an operating room, but adaptable for other settings in the present invention. It includes a plurality of medical devices, each device of which is connected through a single communication channel to the network, wherein each device may be controlled through a local interface, or through a remote interface available through the network. Devices may be readily added or removed from the network without disruption of network functionality. One implementation employs the Jini™ networking protocol (as developed by Sun Microsystems), a description of which may be found at hftp://www.sun.com/jini (dated Sep. 24, 2001), incorporated herein by reference. The Jini network protocol allows a Jini compatible device to make and break network connections instantaneously upon physical connection and disconnection of the device to the network. Further, communications established in a Jini compatible network allow prompt sharing of information between, and control of, devices after connection. This control of networked devices can be orchestrated through standard Internet and web technology such as the hypertext transfer protocol (e.g., hftp over TCP/IP). Jini networking protocol and devices can also be used at a remote facility such as a subject's home to network devices associated with the present invention.

Turning now to the generation of the biosensor signal(s), one or more biosensors measures one or more analytes related to the health of a subject (in many cases, a patient). The medium that may contain the targeted analyte can be withdrawn or collected from the subject's body, such as an analyte in a body fluid or biological sample, or can be in a material to be ingested or taken in by the body of the subject, such as in drinking water, a food to be consumed, or a medication to be applied (e.g., orally or intravenously). An analyte from the subject's body can be obtained by collection of a body fluid or biological sample that is invasively withdrawn (e.g., blood or spinal fluid) or collected after passing outside the body of the subject. The analyte need not be removed from the body of the subject, as in where a measurement is made on or through the skin or other tissues of the body, such as optical measurement of a substance in the blood. In one embodiment, the analyte can be noninvasively withdrawn through unbroken skin or mucosal membranes by noninvasive electro-osmotic withdrawal, as disclosed in U.S. Pat. No. 6,059,736, “Sensor Controlled Analysis and Therapeutic Delivery System,” issued May 9, 2000 to R. Tapper, incorporated herein by reference. They can also be used to momentarily or continuously contact a body fluid or body fluid source.

A biosensor can be in contact with the body or in fluid communication with the body. It can be placed on or adjacent to the skin or other member of the body (generally in fluid communication therewith), in an orifice of the body, inside the body (e.g., a surgically implanted device or a device that is swallowed or introduced by a catheter), in an article that is worn next to the body, and so forth. Biosensors or components thereof can be attached to the skin with hydrogels, including poly(2-hydroxyethyl methacrylate) (PHEMA), whose methods of preparation are described, for example, in A. C. Duncan et al., “Preparation and characterization of a poly(2-hydroxyethyl methacrylate),” European Polymer Journal, Vol. 37, No. 9, September 2001 (published Jul. 6, 2001), pp. 1821-1826.

Biosensors can be spaced apart from the body, such as a biosensor measuring compounds in human breath (e.g., an electronic nose) or other body odors, where they can be in vapor communication with the body. Biosensors spaced apart from the body also include those measuring material removed from the body for separate analysis, such as a blood sensor measuring analytes in withdrawn human blood. Such biosensors can be at any distance from the body, while odor sensors and the like generally should be within a predetermined distance from the body of the subject such as within 15 inches of the body or within 6 inches or 3 inches of the body (i.e., within 6 inches or 3 inches of the closest source of the analyte being measured). In one embodiment, the biosensor (particularly the sensing element thereof) is at least 1 inch away from the body, more specifically at least 3 inches away from the body.

Biosensors can be placed in disposable absorbent articles such as diapers, disposable training pants such as HUGGIES® Pull-Ups®, bed pads, sanitary napkins, panty liners, tampons, interlabial devices, colostomy bags, breast pads, incontinence devices such as incontinence pads, briefs or undergarments. They can also be placed in other devices for collection or disposal of body fluids and other biological waste matter, as exemplified by the flexible waste bags described in WO 00/65348, which can be flexible receptacles for the containment of excreted fecal matter or urine, and in waste receptacles for diapers or other disposable materials, bedpans, toilet bowls, vomit bags, and the like. Biosensors can be associated with an article of clothing such as a shirt, underwear, a vest, a protective suit, an apron or bib, a hat, socks, gloves, or a disposable gown (particularly for medical or surgical use, or for use by a patient), or can be associated with any other object that can be in contact with or near the body, such as a pillow, bed linens, a mattress, breathing tubes, a helmet, face masks, goggles, article of jewelry such as a bracelet or necklace, an ankle bracelet such as those used for prisoners or those on probation, and the like. They can also be physically associated with a wide variety of other objects, such as suppositories, tongue depressors, cotton swabs, cloth towels or paper towels, spill cleanup bags, desiccant bags, disposable mops, bandages, wipes, therapeutic wraps, supports, disposable heating pads, articles of furniture, food containers, and the like.

In specifying where a biosensor is placed, it is understood that not all of the biosensor assembly must be so placed, but that a sensing component thereof is placed in the described location to facilitate measurement. Thus, a sensing element may be placed in a diaper, while other components of the biosensor, such as a power supply or calibration element, may be located elsewhere.

Sampling of body fluids for biosensor detection can be achieved, when needed, by use of the absorbent articles described above. Blood samples and other biological samples can be obtained by any suitable means. Further, for collection of fluids such as saliva, articles with which a saliva sample can be taken, such as a tooth brush, lip stick, lip balm, toothpick, disposable wipe such as a cloth or nonwoven material, and the like can be used.

The biosensor may be in the form of dedicated hardware for repeat uses, or can be an inexpensive, disposable probe for single use or a small number of repeat uses. The biosensor can be incorporated into an article of clothing or disposable article, and can include any of the biosensor technologies and configurations disclosed in the following U.S. patent applications: Ser. No. 09/299,399, filed Apr. 26, 1999; Ser. No. 09/517,441, filed Mar. 2, 2000; and Ser. No. 09/517,481, filed Mar. 2, 2000, each of which are incorporated herein by reference, the contents of which are believed to have been published at least in part in WO 00/65347, published Nov. 2, 2000 by Hammons et al.; WO 00/65348, published Nov. 2, 2000 by Roe et al.; and WO 00/65083, WO 00/65084; and WO 00/65096, each published Nov. 2, 2000 by Capri et al. The biosensor can also include any of the technologies disclosed in U.S. Pat. No. 6,186,991, issued Feb. 13, 2001 to Roe et al., incorporated herein by reference, and in the U.S. patent applications Ser. No. 09/342,784 and U.S. Ser. No. 09/342,289, both filed Jun. 29, 1999 in the name of Roe et al., both of which are incorporated herein by reference, and both of which are related to the disclosure published as WO 01/00117 on Jan. 4, 2001. The biosensor can also be any of those disclosed in U.S. Pat. No. 5,468,236, issued to D. Everhart, E. Deibler, and J. Taylor, incorporated herein by reference. Additional biosensor technologies and systems are set forth hereafter in this document.

Biosensor signals may be continuous or discrete, and may be taken over a short period of time such as a single measurement from one biological sample, multiple measurements over a period of hours or days, continuous measurement during a prolonged period of time such as a year, and the like. Details for the analysis and use of the signals so generated in the context of a healthcare network are set forth hereafter.

More specifically, the invention provides a healthcare network for sharing information concerning the health of a user with at least one outside source, the network including a biosensor associated with the user that generates a biosensor signal containing the information; and a personal data control means including receiving means for receiving the biosensor signal, input means for receiving a privacy input from the user, and output means for generating a response signal based on the biosensor signal and privacy input. The network also includes a data allocation and processing module including means for receiving the response signal, and means for generating and directing an output signal to the at least one outside source, wherein the, module is responsive to the response signal, and wherein the availability of the information to the at least one outside source is responsive to the privacy input.

### BRIEF DESCRIPTION OF THE DRAWINGS

The present invention will be more fully understood and further advantages will become apparent when reference is made to the following detailed description of the invention and the accompanying drawings. The drawings are merely representative and are not intended to limit the scope of the claims.

FIG. 1 is a flow chart illustrating one embodiment of a health care network including biosensors, according to the present invention.

FIG. 2 is a flow chart illustrating further details of the personal data control means of FIG. 1.

FIG. 3 depicts one method for secure connection of a private network to a remote network via the Internet.

FIG. 4 depicts a network configuration for providing restricted access of biosensor information to physicians and other parties.

FIG. 5 is a block diagram of an alternate embodiment of a biosensor network according to the invention.

### DETAILED DESCRIPTION OF THE PREFERRED EMBODIMENTS

As used herein, the term “analyte” means an atom, ion, molecule, macromolecule, organelle, or cell, or, optionally, a mixture thereof, that is detected and measured. The term “analyte” also means a substance in a medium including, but not limited to molecules such as proteins, glycoproteins, antibodies, antigens, hemoglobin, enzymes, target molecules that bind to or react with specific enzymes or other proteins, metal salts, ions (e.g., hydrogen ions, hydroxy ions, sulfates, sulfonates, phosphates, nitrates, nitrites, or electrolytes such as sodium potassium, lithium, or calcium ions), fatty acids, neurotransmitters, hormones, growth factors, cytokines, monokines, lymphokines, lipocalins, nutrients, sugars, receptors, nucleic acids, fragments of DNA or RNA, and pharmaceutical agents or derivatives or metabolites thereof. The term “analyte” also means structured elements such as macromolecular structures, organelles and cells, including, but not limited to cells of ectodermal, mesodermal, and endodermal origin such as cells, blood cells, neural cells immune cells, and gastrointestinal cells, and also microorganisms, such as fungi, viruses, bacteria and protozoa, or characteristic compounds produced by the same. For example, in pH measurement, the analyte can be hydrogen ions and/or hydroxy ions. Some analytes indicate a possible disease condition by either a higher or lower than normal level.

As used herein, “medium” and “biological sample” can refer to any material that can contain an analyte to be measured. A medium or biological sample can be any body fluid, including blood or any of its components (plasma, serum, etc.), menses, mucous, sweat, tears, urine, feces, saliva, sputum, semen, uro-genital secretions, gastric washes, pericardial or peritoneal fluids or washes, a throat swab, pleural washes, ear wax, hair, skin cells, nails, mucous membranes, amniotic fluid, vaginal secretions or any other secretions from the body, spinal fluid, human breath, gas samples containing body odors, flatulence or other gases, any biological tissue or matter, or an extractive or suspension of any of these.

As used herein, “biosensor,” following the definitions given in the CancerWeb Online Medical Dictionary at www.graylab.ac.uk/cgi-bin/omd?biosensor, refers to any sensor that collects data about a biological or physiological process. Biosensors can include any probe, such as those including biological material, which measures the presence or concentration of analytes such as biological molecules, biological structures, microorganisms, etc., by translating a biochemical interaction with the probe into a physical signal. More specifically, the term can refer to the coupling of a biological material (for example, enzyme, receptor, antibody, whole cell, organelle) with a microelectronic system or device to enable rapid low level detection of various substances in body fluids, water, and air.

As used herein, a “biosensor signal” refers to a quantitative or qualitative measurement reading provided by a biosensor, which, without limitation, can be in the form of any of the following:

electronic data, either a digital or analog signal (such as electrical current or a voltage generated directly by the biosensor or indirectly by another device in response to a biosensor reading) that can in turn result in a display on an output device or in data being transmitted to a computer;

a visual cue such as a color change or altered position of an indicator needle or other visible indication of qualitative or quantitative information on devices such as liquid crystal panels, LED arrays, “electronic paper,” or a visible computer display of text or a static or animated image;

a sound such as a beep, a synthesized voice or a prerecorded message;

a temperature change induced by the biosensor; or

any other suitable means of generating a signal to convey information about a measurement made by the biosensor.

As used herein, “medium” can refer to any material that can contain an analyte to be measured. A medium can be any body fluid, including blood or any of its components (plasma, serum, etc.), menses, mucous, sweat, tears, urine, feces, saliva, sputum, semen, uro-genital secretions, gastric washes, pericardial or peritoneal fluids or washes, a throat swab, pleural washes, ear wax, hair, skin cells, nails, mucous membranes, amniotic fluid, vaginal secretions or any other secretions from the body, spinal fluid, human breath, gas samples containing body odors, flatulence or other gases, any biological tissue or matter, or an extractive or suspension of any of these.

As used herein, a “mobile biosensor” is one that can move freely with the subject while a time-dependent biosensor signal (i.e., a time series) is being generated without causing significant disruption or loss of the biosensor signal. A mobile biosensor generally includes a sensing element that is attached to or associated with the person of the subject, such as a sensor in an article of clothing, an absorbent article, or one attached to the skin or implanted in the subject. A mobile biosensor can include signal transmission means such as wireless transmission to receive and process the biosensor signal either continuously or periodically, and/or can include storage means such that a time series of the biosensor signal is stored for later retrieval and processing. Small batteries or other power sources may also be part of the biosensor, when a power source is needed. Biosensors may be small and portable but outside the scope of “mobile” as used herein. For example, a simple pH test strip in a diaper that gives a one-time indication of pH in urine is portable, but not mobile as used herein because it does not provide a time series of pH values.

As used herein, “treatment means” can be any means for delivering a medication, nutritional substance, medical therapy, or other physical or medical care to the subject. Surgery is within the scope of the term. It can also include manual activity, such as turning over a patient in a bed in response to biosensor-detected indicators suggestive of bedsore development or changing a wound cover. It can include administration of a physical treatment such as application of light (e.g., ultraviolet light, simulated sunlight, infrared light, and the like), radiation (e.g., microwave therapy, nuclear radiation, and the like), application of an electrical pulse, movement of the subject, change of a disposable or durable article such as a bed pad or linens, and the like. Oxygen gas or other non-pharmaceutical agents may also be administered.

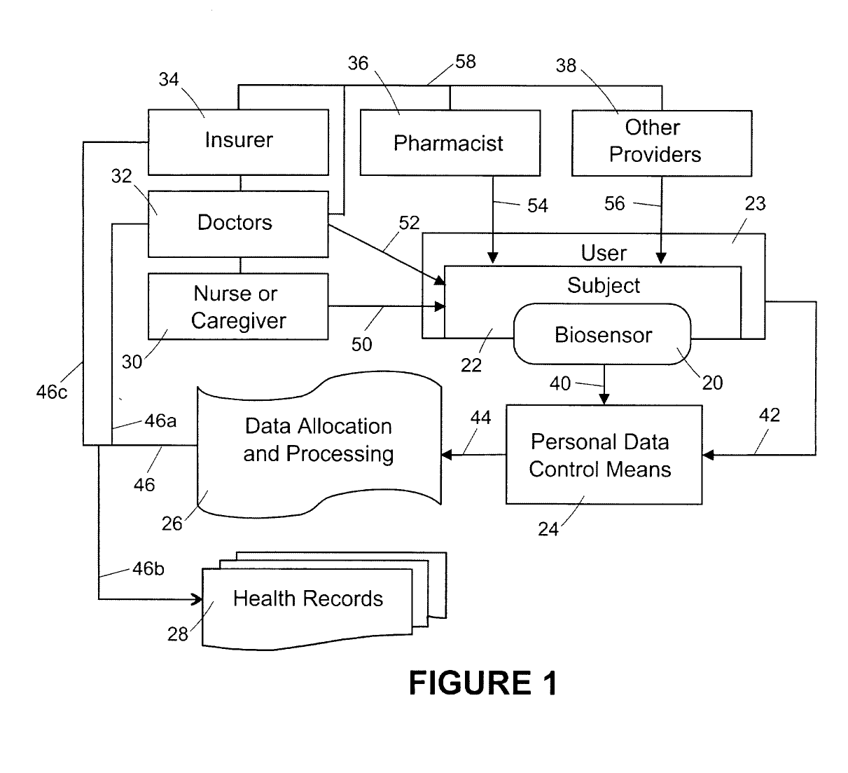

One aspect of the present invention is depicted in the flow chart of FIG. 1. One or more biosensors 20 associated with a subject 22 generates a biosensor signal 40 which is received by a personal data control means 24. The personal data control means 24 can include a computer or microprocessor, software for acquisition and interpretation of biosensor data, data acquisition means, a display system such as a monitor, and user input means to allow the user 23 (either the subject 22 or a representative of the subject 22) to provide a privacy signal 42 to specify how the data or results from the data can be shared with others and/or to provide a means for annotating biosensor results. In one embodiment, the personal data control means 24 can include a programmable portable data acquisition and display device such as a personal digital assistant (PDA) equipped to receive a wireless signal from a biosensor and then provide a display to the user 22 showing a preliminary assessment of the meaning of the signal, whereafter the user 23 can then choose to transmit the data for review by a doctor or other expert and can specify whether the information will be available to other parties such as an employer or insurance agency (though in one embodiment, the outside source can be informed of the user's refusal to forward health-related data from the biosensor). In another embodiment, the data acquisition and display device can be an I-STAT portable clinical analyzer from i-STAT Corporation (East Windsor, N.J.) or a modification thereof. Two or more electronic devices cooperatively associated, such as a data display device and a user input device can be used. A wide variety of electronic dataloggers can be used as a component of the personal data control means 24 for receiving and storing a biosensor signal 40 over a period of time and then optionally computing and displaying results from the accumulated biosensor signal 40, or transferring the data to another device for optional computation and display of an interpretation of the data for review by the user 23 or other party. Exemplary dataloggers include the cable and wireless dataloggers of Ellab A/S of Denmark (with offices in San Jose, Calif.), and other suitable dataloggers. Smart cards can also be used, as described more fully below.

The personal data control means 24 combines the biosensor signal 40 and a privacy input 42 in generating a response signal 44 that is sent to a data allocation and processing module 26, which may be physically remote from the personal data control means 24 or may be adjacent to or integrated with the personal data control means 24. The data allocation and processing module 26 can include a central server or other computer database means where data from the biosensor 20 may be allocated (selectively distributed) for use by outside parties and for optional storage in health records 28. In one embodiment, the data allocation and processing module 26 is on a server of a private network remote from the subject 22 and the personal data control means 24.

The data allocation and processing module 26 handles the allocation of health-related data pertaining to the subject 22 in response to the response signal 44. One or more output signals 46 are generated and directed to one or more outside sources, including a signal directly sent to doctors 32 or other health professionals 46a, which can be shared in whole or part with nurses or other caregivers 30; personal health data 46b that can be entered into personal health records 28, either in electronic form (e.g., a searchable, archived record with restricted access) or as a printed record entered into a file, or both; and other information 46c suitable for use by an insurer 34 or other agency, an employer, or the like. Portions of the data in the output signal 46 may be interpreted and combined with other information to result in instructions being given to a pharmacist 36, nurse or caregiver 30, insurer 34, other providers of health care services 38, and other parties, preferably in compliance with best medical practices. These parties can in turn provide services or treatments 50, 52, 54, 56 for the benefit of the subject 22 in response to the instructions received. The data allocation and processing module 26 can include storage means (e.g., a tape backup, hard disk, floppy disk, and other means) to store the response signal 44 or information derived therefrom, including the storage (archiving) of medical records including data derived from the response signal 44.

Access to biosensor-derived information in the health records 28 may be partly restricted or coupled with annotations from the user 23 or other party, in response to the privacy input 42. The same applies to all other uses of biosensor-derived information.

The response signal 44 can be transmitted to the data allocation and processing module 26 by a radiofrequency signal, infrared (IR) signal, electronic signal over a cable or wire (e.g., an Internet connection, a phone line, and so forth), optical signal over a fiber optic cable or other means, and the like.

After data allocation and processing 26, portions of the data (or all the data) can be sent to a doctor 32, who can share it with nurses or other caregivers 30 to guide the actions taken to care for the subject 22. Portions of the data may be shared with insurers 34 or other agencies or institutions (e.g., the Center for Disease Control, or the National Institutes of Health), as determined during data allocation and processing 26. Recommendations regarding medication, for example, may be made by doctors 36 and payment authorization therefor may be provided by an insurer 34, resulting in an order sent to a pharmacist 36 or other providers 38 to prepare materials required for care of the subject (e.g., drugs or other medical aids). A new treatment or change in treatment may be administered to the subject 22, and the biosensor can again be used to track the efficacy of the treatment. The treatment may include not only changes in therapy, diet, medication, and the like, but also may include a recommendation for one or more additional biosensors or for a new biosensor to monitor additional analytes or biological processes.

A doctor may be authorized to review current biosensor data and past medical records and biosensor data. Depending on options selected by the user 23, the doctor may then adjust medications or other services provided to the subject 22 based on information from the biosensor. Some information may be directly shared with a nurse or other caregiver, and the insurer, who may need to be apprised of medical needs and recent sensor readings in order to authorize coverage for some services. Decision made by doctors in light of the biosensor data may be used to direct a change in prescription drugs or other care services provided to the subject 22. Authorization from insurers may be obtained, either manually or via an automatic electronically generated request. The ability of a doctor to reliably alter medication based on remote biosensor data may require that the identity of the user 23 be authenticated through methods such as biometrics or multi-factor authentication, and may require the user 23 to waive some levels of privacy protection to ensure that transmitted data is complete and accurate.

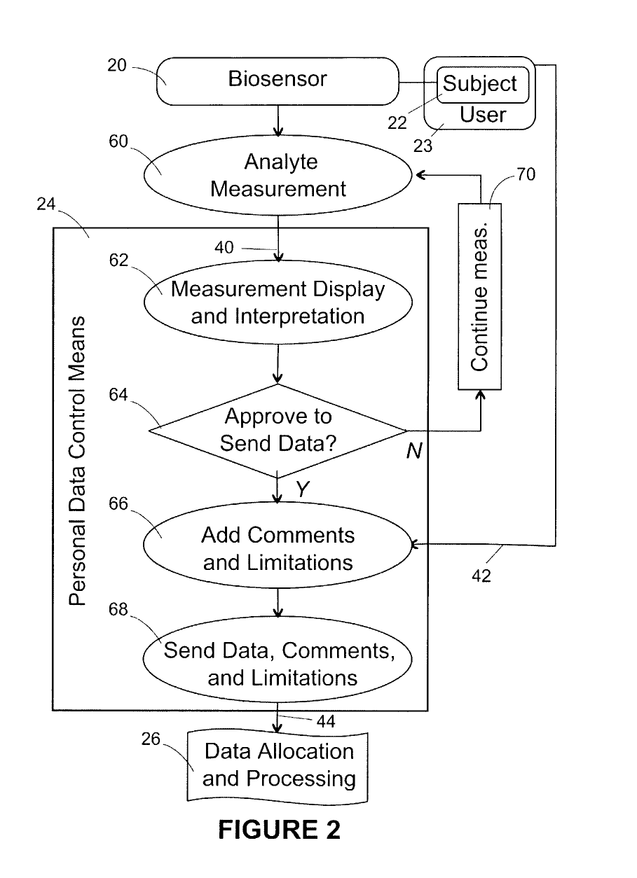

FIG. 2 shows additional details associated with the personal data control means 24. A biosensor 20 interacts with a subject 22 to yield an analyte measurement 60 conveyed via a biosensor signal 40. The biosensor signal 40 can be read by an electronic display device for measurement display and interpretation 62. A portable device or computer may receive the signal and generate a reading that can be interpreted by the user 23. For example, the display may show that the analyte level is abnormal or potentially indicative of a pathological condition, or it may show that a possible malfunction has occurred. Alternatively, a datalogger, smart card, or other device may record and store the biosensor signal, which later can be reduced or manipulated for display to the user 23, either by circuitry and display features integral with the datalogger or smart card, or with the assistance of one or more additional devices.

The user 23 is then provided with an opportunity to send the data to a data allocation and processing module 26. If approval is not given, measurement can continue 70 to provide further opportunities for measurement.

When approval is given, the user 23 can be prompted to provide a privacy input 42 to add comments and limitations 66 regarding the use of the data, or circumstances relating to the data, or other information that can assist in properly interpreting the data and protecting the privacy of the subject 22. Data from the biosensor signal 40 and the privacy input 46 are combined in a response signal 44 that is transmitted to a data allocation and processing module 26.

For example, a smart card such as the Data Concern TM Smart Card marketed by Lifestream Technologies (Post Falls, Id.) can be used to store cholesterol information provided by a biosensor signal 40 from a Lifestream Technologies® Personal Cholesterol Monitor taken over a period of time. The Data Concern™ Smart Card can then be used to provide data for display on another device than can also provide input means for the user 23 to enter a privacy input 42. A smart card can be used with non-volatile memory, including FRAM (ferroelectric RAM). The Data Concern TM Smart Card utilizes a microprocessor and Microsoft® Smart Card for Windows operating system. Optionally, the Data Concern™ Smart Card can be combined with the Privalink™ software package to add emergency medical information directly to a Personal Health Card™, including drug and food allergies, prescriptions, insurance company, primary care physician and hospital preference, as well as other critical information. Physicians and pharmacists can be authorized to access test results and personal health records over the World Wide Web. It is within the scope of the present invention to adapt such smart card systems to issues other than cholesterol monitoring, including hormone monitoring during hormone replacement therapy (HRT); clotting time (prothrombin time) monitoring during anti-coagulant therapy; thyroid hormone monitoring during therapy; and blood pressure monitoring during anti-hypertensive therapy. Another system of potential value is disclosed in WO 00/52457, “Card-Based Biosensor Device,” issued to W. Y. Wong et al. of Helix Biopharma Corp., Canada.

Measurement display and interpretation 62 typically results in an intermediate output signal that is displayed for reading or interpretation by the user 23 or other caregiver. The intermediate output signal can include qualitative or quantitative results displayed on a screen or other display device in the form of text, a bar graph, a numerical value, a pie chart, an icon, a color, and so forth, or can be a sound such as a synthetic voice, a beeping of variable frequency or intensity, a vibration of a physical device, and the like. Detailed display of information with interpretative guidance on a computer screen or the like with live hypertext for additional information represents one embodiment for the intermediate output signal. A display responsive to measurement by a biosensor 20 can also employ electrochromic inks, wherein a displayed color is related to an applied voltage. Information on electrochromic inks is available at composite.about.com/library/PR/2001/blufl1.htm, unisci.com/stories/20012/0515016.htm, and I. Schwendeman, et al., “Combined Visible and Infrared Electrochromism Using Dual Polymer Devices,” Adv. Mater., Vol. 13, 2001, pp. 634-637, and B. C. Thompson et al., “In Situ Colorimetric Analysis of Electrochromic Polymers and Devices,” Chemistry of Materials, Vol. 12, No. 6, June 2000, pp. 1563-1571. Display of information can also occur with LCD screens or other LDC displays, or with “electric paper” such as that described in U.S. Pat. No. 6,284,352, “Ferrofluidic Electric Paper,” issued Sep. 4, 2001 to Biegelsen et al., incorporated herein by reference. Colorimetric film can also be used, in this application as well as in direct response to an analyte or signal generated by a sensing element, including the use of calorimetric film described in U.S. Pat. No. 6,001,556, incorporated herein by reference.

In general, the personal data control means 24 permits the user 23 or other party to control what information (e.g., a subset of the data derived from the biosensor signal 40) is transmitted to other parties, and/or to whom it is transmitted, and/or what additional information (such as user comments or explanatory notes) is sent with the data. The response signal 44 from the personal data control means 24, as well as the output signal 46 from the data allocation and processing module 26, can be encrypted. Encryption of data for security can be by any suitable means, including methods based on chaos theory such as fractal-based encryption (see, for example, “Fractal-based Encryption,” Photonic Tech Briefs, Vol. 25, No. 7, July 2001, pp. 14a-16a), including the methods provided by Catnaz, Inc. of Columbus, Ohio.

The output signal 46 can also include unique identification information such as a user ID and password or PIN from the user 23 or from each person modifying the data or adding comments. The serial number of one or more devices associated with the personal data control means 24 or other hardware-related identifying information can also be sent, as well as identifying information pertaining to the biosensor 20 (e.g., a product code conveyed via an RFID or smart tag system) or other data signals (not shown) such as a personal identification code for the subject, signals from temperature sensors and other sensors, and the like. Unique registered ID labels for each biosensor 20 or for other devices associated with the biosensor 20 can be included in the signal sent to the data allocation and processing center 26 to track specific sensors and ensure that proper equipment is used or that equipment signals are not falsified.

The personal data control means 24 optionally can provide additional feedback to the user 23 about how transmitted data have been used. The user 23 can select, for example, to permit a hospital or doctor to continuously observe the biosensor signal 40, or can choose to transmit data derived from the biosensor signal 40 periodically or at arbitrary intervals selected by the user 23. The user 23 may wish to not transmit some data, especially when there was a problem such as temporarily disconnecting the biosensor 20 from the user 23. Or the user 23 may choose not to transmit data for other reasons. For the best health care, the data should be readily available to primary care providers. The method of integrating a biosensor 20 to a health care system may also include the step of providing electronic confirmation to the user 23 that a transmission of data has occurred, and separately indicating when and by whom the data has been reviewed. Thus, after biosensor data have been transmitted to a doctor, for example, the user 23 can know how long before a doctor saw and considered the data (or considered a computer-generated analysis of the data). Further, the patient may be electronically provided with the doctor's comments on the biosensor data and with his or her planned course of action in response. The patient may then have the option to challenge the planned course of action or call for a second opinion before accepting adjustments in treatment.

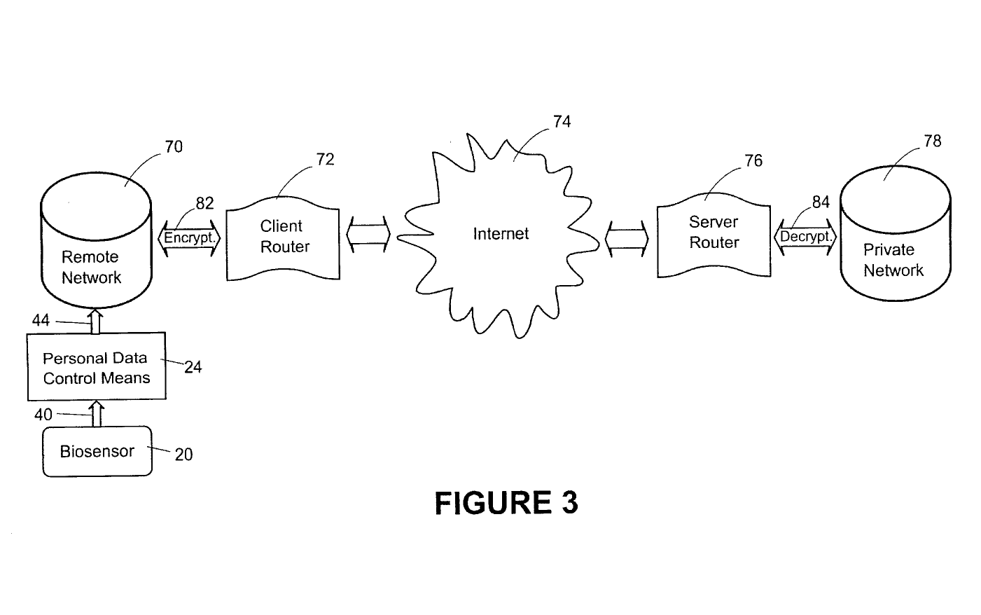

FIG. 3 shows one system for sharing of information from a remote biosensor 20 with a central network in a way that protects the security of the data. The response signal 44 from the personal data control means 24 provides data to a remote network 70, which can include a lone data transmission device that can be part of the personal data control means 24. The remote network 70 provides the data in the form of a signal to a client router 72, with an intermediate encryption step 82 occurring to encrypt the data. The encryption step 82 can also include decryption of a signal received from another source via the client router 72. The client router 72 directs a signal including the encrypted data over the Internet 74 to a server router 76, which provides the signal to a private network 78 with an intermediate decryption step 84. The decryption step 84 can also include encryption for a signal sent from the private network 78 to another source such as the remote network 70. The private network 78 can form part or all of the data allocation and processing module 26 (not shown). In this process, a secure tunnel can be provided between the client router 72 and server router 76, as explained at www.linuxdoc.org/HOWTONPN-HOWTO-2.html#ss2.1. To establish the secure tunnel, any suitable method can be used, including Point-to-Point Tunneling Protocol (PPTP).

Secure transmission of data to authorized recipients can be achieved using Microsoft's Platform for Privacy Preferences, or P3P (see, for example, “The Battle Over Web Privacy” by G. R. Simpson, Wall Street Journal, Mar. 21, 2001, pp. B1, B4). User settings determine the level of privacy, and can be adapted more specifically for the needs of the present invention.

The system of WO 01/39021 can also be used. This describes an interactive system for transferring and submitting information, having: an external user interface; an external content administrator in communication with an external submission data store and external user interface, wherein the external content administrator includes executable instructions for collecting technical information from an external user; an internal content administrator in communication with an internal submission data store and the external content administrator, wherein the internal content administrator includes executable instructions for processing technical information from the external user; and a security module, wherein the security module includes executable instructions for limiting access between the external user interface and the internal submission data store through the external content administrator.

The remote network 70 can include or be part of the family information management system and related database structures proposed in WO 00/77667 by S. E. Young et al., published Dec. 21, 2000, which claims priority from U.S. patent application Ser. No. 60/139,111, filed Jun. 14, 1999, incorporated herein by reference.

Biosensors 20 tied to care networks may be used for numerous purposes, including:

detecting the onset of infection or the status of an infection for a recovering patient;

monitoring the health of fetus or mother during pregnancy (pregnancy management), detecting such things as premature delivery by monitoring uterine contractions, antiphospholipid antibodies, fetal fibronectin proteins, and so forth;

monitoring reproductive status (e.g., onset of ovulation or other factors associated with fertility);

other hormone detection (e.g., growth factors, thyroid, menopause-related ones, etc.)

detecting the onset of menstruation;

monitoring analytes associated with renal disease, including analytes in the blood or urine measured before, during, or after dialysis, and analytes measured in any body fluids at home or for patients not receiving dialysis,

monitoring risk factors for osteoporosis, or the onset or status of the disease, or hormone levels or other agents correlated with the development or treatment of osteoporosis and other bone pathologies, through means such as monitoring bone-specific alkaline phosphatase or calcitonin;

monitoring factors related to heart disease, including analytes such as myoglobin, troponins, homocysteine, creatine kinase, thrombus precursor protein, fatty acid binding protein, CRP, and the like;

monitoring factors related to rheumatoid arthritis, including MMP-3, fibrin degradation products, anti-type II collagen, and collagen cross-linked N-telopeptides;

detecting factors related to stroke, including D-dimer in the blood or other body fluids;

monitoring the effectiveness or presence of a pharmaceutical agent such as an antibiotic;

detecting an enzyme or other factor associated with heart disease to alert a patient and/or care givers of a potential cardiovascular problem;

identifying rheumatoid arthritis by detecting type I collagen crosslinked N-telopeptides in urine;

monitoring cyanosis or circulatory disorders in newborns, diabetics, and so forth;

monitoring the onset of a sleep apnea episode, coupled with treatments to enhance sleep when needed; such a concept could include the system disclosed in WO 99/34864, published Jul. 15, 1999 by N. Hadas, the U.S. parent of which is incorporated herein by reference;

optically monitoring nail beds as a tool for assessing blood condition (for some tests, nails can be more transparent than skin to changes such as bluing);

tracking body position in a bed and applied pressure against the skin of the patient in order to prevent or care for bedsores (decubitus ulcers) and other ulcers or wounds (one means for tracking applied pressure includes the printed arrays of pressure detecting films marketed by Tekscan, Inc. of South Boston, Mass., which can serve as a sensor indicating pressure applied by the body to various points under the body; videocameras, load cells, and other tools can also be employed for tracking position and load; and position detectors can monitor the level and position of the bed over time to ensure that patient position is regularly adjusted); biosensors indicative of wound health and protein-degrading enzymes can also be employed in cooperative association with pressure and position sensors for this purpose;

tracking indicators of health by monitoring of body odors or analytes in the gas phase near the body, using electronic nose technology or other sensors;

tracking stress with cortisol measurement in saliva or seratonin measurement, including establishing moving baselines to distinguish between acute stress and chronic stress, and optionally relating the time history of measured stress-related analytes to factors that may have induced the stress;

using archived time histories of one or more analytes as a record for identification of sudden changes in the treatment of a subject that may be traceable to changes in personnel, medication, and the like, wherein the time history may serve as a tool in detecting malpractice or other problems, or in verifying (or refuting) claims made by the user regarding health status of the subject;

detecting allergies using as analytes any of IgE (immunoglobulin E), eosinophilic cationic protein, cytokines such as IL-4 or IL-5 in mucous or in the blood or other body fluids, including the use of facial tissue equipped with biosensors for such analytes or with biosensors for bacteria or virus infection;

detecting bacterial infections using analytes such as cytokines (e.g., IL-6), C-reactive protein, calcitonin or pro-calcitonin, CD11b, ESBL enzymes (particularly for drug-resistant bacteria), and lipocalins;

detecting risk factors for cervical cancer by monitoring nuclear matrix protein (NMP) 179 or human papilloma virus from a pap smear;

monitoring levels of taurine in the body or in a local region, including monitoring taurine levels in a non-human mammal such as a domestic cat;

urinary tract infection testing;

yeast infection, bacterial infection, or other forms of vaginitis, including pH imbalance;

UV exposure detection;

nutritional monitoring or detection of nutrient levels, also including hydration monitoring, cholesterol testing, energy assessment, and anemia assessment;

monitoring of pesticides, preservatives, and other harmful compounds in a food product (e.g., milk produced from cattle in a dairy operation, or in food to be consumed by humans), including, for example, a biosensor based a cotton cytokinin receptor, as disclosed by V. V. Uzbekov et al., “Chemical Modification of Components of the Cotton Cytokinin Hormone-Receptor Complex for Creation of Pesticide Biosensors,” Chem. Nat. Compd. Vol. 36, No. 6, 2001, pp. 611-615;

measurement or monitoring of stress indicators;

allergy testing or detection of allergens;

detection or screening for ear infection;

cardiovascular/respiratory health (including pre-heart attack detection, post heart attack detection/monitoring, overall heart health, oxygenation monitoring, pulse, heart dysrythmia alert, respirations, stroke detection, pneumonia detector, respiratory differential, sleep apnea detection);

detection of influenza with devices such as the FLU OIA™ biosensor of Thermo BioStar (Boulder, Colo.), or detection of other diseases with Thermo BioStar biosensor materials, or with other methods such as lateral flow analysis, diffraction-based methods, electrochemical detection;

musculoskeletal testing (muscle performance, osteoporosis, body fat);

monitoring health factors in neonates, such as bilirubin levels for jaundice detection; and

monitoring blood sugar levels for diabetics; and so forth, as set forth in more detail below.

The biosensor 20 may provide measurements in real time, measurements at periodic intervals (e.g., snapshots in time), time-averaged results, and the like. The biosensor 20 can be worn on the body or against the body. By way of example, it may be placed inside or on an absorbent article such as a bed pad, a diaper, a sanitary napkin, facial tissue, ostomy bag, tampon, disposable garment, incontinence product, and so forth. It can also be an electrode, optical device, or other instrument, preferably miniaturized, that can respond to health indicators from the subject's body. The biosensor 20 may detect one or more analytes directly. Any suitable biosensor technology can be used, including dielectrophoresis, free-flow electrophoresis, ATP bioluminescence, DEFT, impedance, LAL, ELISA and other immunoassay methods, pH measurement, optical diffraction-based techniques, agglutination techniques, chromogenic agars, molecular imprinting for the real-time analysis, and the like.

Analysis of the detected signal to assess the health of the subject can be based on comparison to fixed parameters or parameters that are adjusted over time. One useful example of the latter approach is disclosed in U.S. Pat. No. 5,555,191, incorporated herein by reference, which describes an automated statistical tracker that can detect malfunctions in equipment. Messages are received from the sensor over a significant period of time to form message subgroups consisting of selected numbers of messages, and the messages in each of the subgroups are compared to predefined units for that subgroup to determine whether the number of messages in that subgroup that are statistically unusual. Thereafter, an alert signal is generated whenever a statistically significant number of unusual signals are detected. Threshold limits for the measurements are automatically calculated and regularly updated, rather than using fixed limits. The data can be fit to normal or Poisson distributions, for example, from which upper and/or lower limits of acceptable messages per time period can be calculated.

In addition to the biosensor signal 40, any number of additional signals (not shown) may be received by the sent to a data allocation and processing module 26. Such signals can be transmitted by any means such as UWB signals, AM or FM radiofrequency signals, direct wiring, the Internet, a modem, and the like. The additional signals can include readings from other sensors providing measurements of factors such as room temperature, light levels, the location of the subject via a signal from a Global Positioning System (GPS) device or other positioning means, information regarding medications received, operational status of therapeutic devices, the presence of others in the room, whether or not the individual is in bed (e.g., using a load sensor in the bed), and the like. In one embodiment, the presence of specified objects or persons near the subject can be detected by detection means and transmitted with or in addition to the biosensor signal to the data allocation and processing module 26 or to another module (not shown) for continuous monitoring of the well-being of the patient.

For example, objects comprising “smart tags” for radiofrequency identification (RFID), such as the smart tags under development at the Auto-ID Center at Massachusetts Institute of Technology (Cambridge, Mass.) can convey a unique electronic product code via a miniature antenna in response to a radio signal from an RFID reader, which can read the code of the object. The object code can be used to determine the nature of the object. In one embodiment, an RFID scanner associated with the subject reads a plurality of objects in the room and transmits the object codes to a processor or other computer-device that can determine if appropriate or inappropriate objects are present. The product code can be sent via the Intranet or other means to a server containing information relating product codes and object descriptions, which can return the information to the processor (not shown) or other device or party for evaluation or recoding of relevant information. Inappropriate objects that could be detected could include a pack of cigarettes, a food product to which the individual is allergic, weaponry or other contraband, a person forbidden to have contact with the individual, or electronic devices unsuitable for a patient with a pacemaker. Appropriate objects could include a humidifier, a wheelchair, a caregiver, an oxygen tank, devices to assist walking, and so forth. An RFID reader can also read a unique ID code from a smart tag or other device associated with the individual or the biosensor or both and the code or codes can be sent to the data allocation and processing module 26.

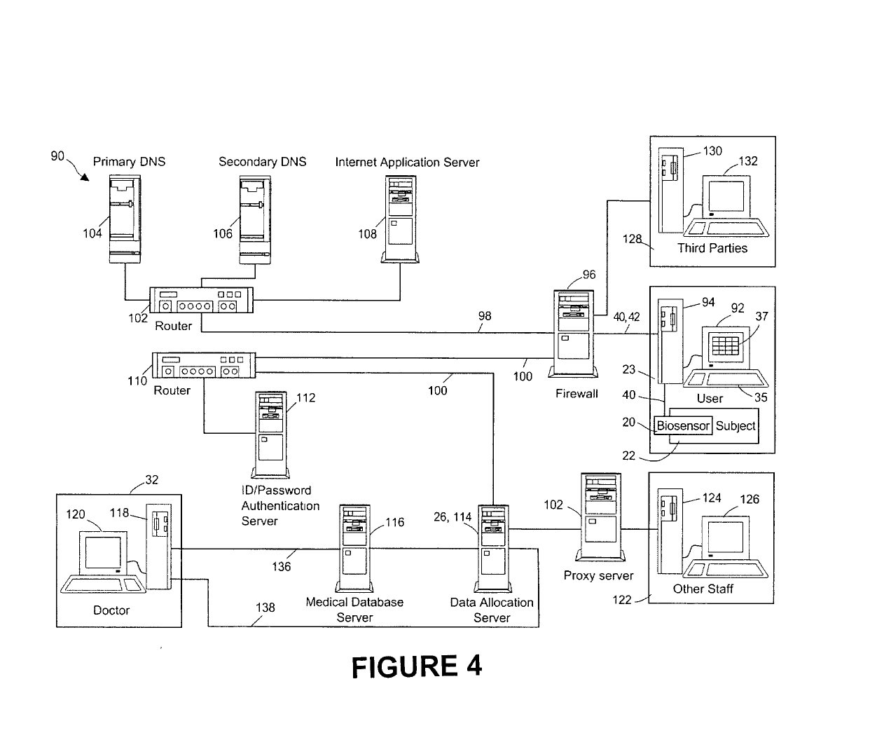

FIG. 4 depicts one embodiment of a computer network 90 supporting the healthcare network of the present invention. Communication between the computer 94 of the user 23 with the computer network 90 can be provided via a Web-based interface beginning. Upon entering a predetermined URL for the Web page, the URL request is sent via the firewall to a Cisco router 102, which employs either a primary domain name server (DNS) 104 or a secondary DNS 106 to determine the IP address to be used for the requested URL. A signal is then sent to an Internet application server 108, which generates a signal to create a Web page display. The signal is routed back to the computer 94 of the user 23 such that a Web page is displayed on a monitor 92. The displayed Web page requires the user to log in using a user ID and password (or other authentication means such as biometrics). When the user ID and password are entered, that information is routed again through the firewall 96 to a second Cisco router 110 that directs the information to an ID/password authentication server 112 (e.g., an SQL server). If a valid user ID and password have been entered, a welcome page for the computer network 90 is then displayed (e.g., a signal is sent to the Internet application server 108 which then sends a signal back to the computer 94 of the user 23 to display the computer network welcome page). The welcome page displayed after logging in is unique to the subject 23 and can provide access to additional pages that contain information unique to the user 23 and/or subject 22, including default settings for access to data and distribution of data, biosensor 20 information such as model type and serial number, insurer information, special directions for emergency response, and so forth. This information can be stored on the Internet application server 108 or a data allocation server 114, and/or the computer 94 of the user 23.

In this embodiment, once the user 23 has been authenticated, access is granted to the data allocation server 114.

The biosensor 20 measuring health-related information from the subject 22 provides a biosensor signal 40 which is received by the computer 94 of the user 23, who can be the subject 22 or a representative of the subject 22. The computer 94 displays an intermediate output signal 37 summarizing the biosensor data over a period of time (variable or predetermined) and optionally indicating problems or a potential diagnosis. The user 23 can enter a privacy input 42 through the keyboard 35 that can be sent with the biosensor signal 40 or information derived therefrom to a firewall 96. The privacy input 42 can be prompted, meaning that the computer 94 of the user 23 issues a request for a privacy input 42 before data are transmitted to the firewall 96. The prompting for a privacy input 42 may occur periodically, such as one a day, or before any data can be transmitted in response to a manual or automatic request to transmit data. Alternatively, a privacy input may be sent within a predetermined time period after data (e.g., 1 hour or 1 day) have been sent to the computer network 90, such that the user 23 can modify access to the data after data have been received but preferably before others have had access to the data.

The privacy input 42 and biosensor signal 40 or data derived therefrom are securely routed from the computer 94 of the user 23 through the firewall 96 and to the data allocation server 114, where software and hardware function as the data allocation and processing module 26. Data from the biosensor signal 40, as determined by the privacy input 42, can then be made available to outside parties. Information may be entered into secure medical records on a medical database server 116. A doctor 32 may receive a signal 138 from the data allocation and processing module 26 indicating that biosensor signal data are available for review, whereupon the doctor 32 may access medical information 136 from the medical database server 116.

The data allocation server 114 and other aspects of the computer network 90 can also be accessed by other medical staff 122 via a proxy server 102. The other staff 122 can include an administrator who maintains the data allocation server 114 and makes any needed corrections.

Other third parties 128 can access portions of the biosensor data or other medical information about the subject 22 using a Web-based system or other means via the firewall 96, allowing data to be received on third-party computers 130 and displayed on third-party monitors 132 for decision making, approval of claims, or other purposes, with access responsive to the privacy input 42 of the user 23.

The privacy input 42 can also include an electronic signature from the user 42, along with data of entry, subject ID, and other information.

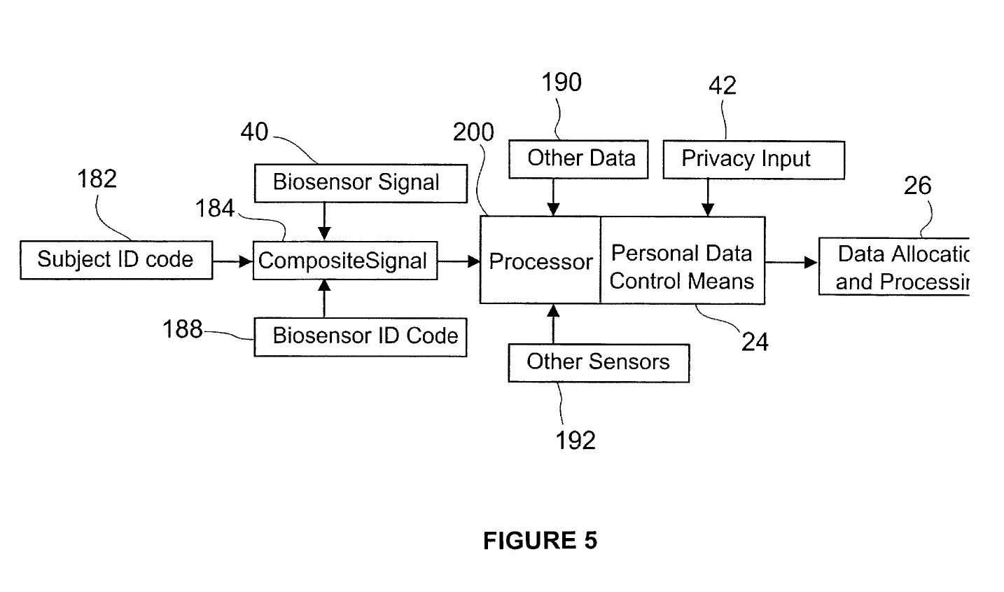

As shown in FIG. 5, biosensor signal 40 can be combined with a subject ID code 182 and a biosensor ID code 188 to form a composite signal 184 which is directed to a processor 200 in control of the user (not shown) and cooperatively associated with personal data control means 24. The processor 200 may also received data from other sensors and other data sources 190, such as annotations or instructions entered by a physician or caregiver, medical history of the patient, insurance status, and so forth. Based on the rules established by the user or the decisions made by the user in directing the personal data control means, the data from the composite signal 184 and other sources 190, 192 can be filtered such that only a subset is available to the data allocation and processing module 26, or such that different components of the information have different levels of access by third parties responsive to the privacy input of the user governing the personal data control means 24.

### a. Biosensor Details

The biosensors used in the present invention can be suitable for use outside of a hospital, such as for home use or use in a managed care facility. While many biosensors require that a skilled medical professional take the reading and/or interpret the results, it is within the scope of the present invention to employ biosensors for which quantitative or qualitative data can be obtained or read by the user and/or family member or caregiver with or without specialized medical training. While interpretation and diagnosis of the data may typically require a skilled medical professional, biosensors can be used that enable the user to understand the nature of a health factor, such as a blood glucose level, and take or request appropriate action in response to the biosensor signal.

Biosensors for any disease or ailment can be considered, including cancer. For example, markers in urine can be detected for bladder cancer (e.g., BLCA-4, a nuclear matrix protein found in the nuclei of bladder cancer cells, a described in Diagnostics Intelligence, v 10, no 5, p.12). Vascular endothelial growth factor and NMP 22 can also be useful analytes. For melanoma, circulating S-100B can be a useful analyte. For prostrate cancer, human glandular kallikrein, prostrate-specific antigen, and E-cadherin can all serve as useful analytes (in the case of E-cadherin, lower levels may be associated with cancer). U.S. Pat. No. 6,200,765, issued Mar. 13, 2001 and incorporated herein by reference, discloses a noninvasive method of detecting prostrate cancer using a body fluid sample, which can be urine. Thus, incontinence products or other absorbent articles could be equipped with biosensors for prostrate cancer, bladder cancer, or other cancers. Feminine care products could also be equipped with biosensors for detecting cervical cancer. One useful marker for cervical cancer is a marker known as NMP-179, (NMP=nuclear matrix protein), which has been linked to cervical cancer by Matritech. Breast epithelial antigen can also be a marker for breast cancer, and has been proposed as an analyte for detection with flexural plate-wave (FPW) sensors. WO 01/20333 discloses a system for cancer detection by detecting midkine in urine or blood. In vitro detection of diseases such as cancer is disclosed in WO 01/20027.

Various types of sensors employing electrical, optical, acoustical, chemical, electrochemical or immunological technologies can serve as biosensors. The can be miniaturized to function as microsensors. The biosensors of the present invention can be disposable (single-use or multi-use devices), or can be durable sensors for repeated use or continuous use over a prolonged period of time. Those based on bioaffinity, biocatalysis, or other operating principles can be used.

Many biosensors include a sensing layer associated with a transducer. The sensing layer interacts with a medium including one or more targeted analytes. The sensing layer can include a material that can bind to the analyte and can be, for example, an enzyme, an antibody, a receptor, a microorganism, a nucleic acid, and the like. Upon binding of the analyte with the sensing layer, a physicochemical signal induces a change in the transducer. The change in the transducer permits a measurement that can be optical (e.g., a viewable diffraction pattern), potentiometric, gravimetric, amperoteric, conductimetric, dielectrimetric, calorimetric, acoustic, and the like.

Many biosensors for particular analytes use ELISA (enzyme-linked immunosorbent assays), wherein specific enzyme-labeled antibodies are employed to detect an analyte. Any suitable ELISA method can be employed herein. Solid-substrate assay techniques are typically combined with colorimetric or fluorescent signals to indicate the presence of the analyte, though gravimetric measurement can also be employed. One such example is given by Amy Wang and Richard White at the Berkeley Sensor and Actuator Center, University of Berkeley, described at buffy.eecs.berkeley.edu/IRO/Summary/97abstracts/wanga.1.html, which discloses the use of flexural plate-wave (FPW) sensor wherein the amount of protein bound to the solid substrate (the flexing plate of the FPW device, a micromachined, acoustic sensor along which ultrasonic flexural waves propagate) is measured by a change in acoustic wave velocity caused by the added mass of the bound proteins. Any other measurement technology can be used. Basic principles of immunological sensors are given in P. Tijssen, Practice and Theory of Enzyme Immunoassay, Elsevier, Oxford, 1985, and D. Diamond, Principles of Chemical and Biological Sensors, Wiley and Sons, New York, 1998. Other principles of biosensors employing antibodies are disclosed in WO 01/27621; WO 01/27626; WO 01/27627; WO 01/20329′ WO 00/08466; and WO 99/64620.

Biosensors can include multiple sensing elements or other technologies to detect multiple analytes. For example, one can employ the multiple analyte technology of U.S. Pat. No. 6,294,392, “Spatially-Encoded Analyte Detection,” issued Sep. 25, 2001 to Kuhr et al. provides a flow-through microfluidic (e.g., capillary) biosensor for detecting different target analytes (e.g. nucleic acids) in a sample after binding to their cognate “binding partners” (e.g. nucleic acids, antibodies, lectins, etc.). In general, binding partner “probes”, specific to various analytes are immobilized in different sections of a capillary channel, e.g. using photolabile biotin/avidin technology. The sample is then flushed through the capillary, so that the target analytes are bound to the binding partners (capture agents) immobilized on the capillary wall and the rest of the sample is eluted from the capillary. Finally, the complexed (bound) analyte is released along the entire length of the channel and flushed past a detector. In a preferred embodiment, the desorbed, target-analytes are detected at a copper electrode poised downstream using sinusoidal voltammetry (Singhal and Kuhr, Analytical Chemistry, Vol. 69, 1997, pp. 3552-3557; Singhal et al., Analytical Chemistry, Vol. 69, 1997, pp. 1662-1668). The time from the elution of the target analyte(s) to detection is used to determine the identity of each analyte. Multiple analytes, of the same species of molecule (e.g., all nucleic acids), or of different species (e.g. proteins and nucleic acids), can be diagnosed by using a single biosensor in this manner. The sensor is said to be highly specific due to the use of specific binding partners, and extremely sensitive due to electrochemical detection.

Numerous techniques exist for immobilizing an enzyme or other bioactive material on a substrate. Recent developments include siloxane-based biocatalytic films and paints, in which enzymes are immobilized by sol-gel entrapment of covalent attachment into a polydimethylsiloxane matrix, as described by Y. D. Kim et al., “Siloxane-Based Biocatalytic Films and Paints for Use as Reactive Coatings,” Biotechnology and Bioengineering, Vol. 72, No. 4, 2001, pp. 475-482. Methods for using polytetrafluorethylene (PTFE) substrates have also been developed to enable PTFE use as a polyfunctional support, as described in M. Keusgen et al., “Immobilization of Enzymes on PTFE Surfaces,” Biotechnology and Bioengineering, Vol. 72, No. 5, 2001, pp. 530-540. Elemental sodium and then ozone or peroxide oxidation is used to open up covalent attachment points for enzyme binding. Enzymes can also be immobilized in silica gels, as described by M. Schuleit and P. Luisi, “Enzyme Immobilization in Silica-Hardened Organogels,” Biotechnology and Bioengineering, Vol. 72, No. 2, 2001, pp. 249-253.

Another useful substrate and biosensor is that of Dieter Klemm and Lars Einfeldt, “Structure Design of Polysaccharides: Novel Concepts, Selective Synthesis, High Value Applications,” Macromolecular Symposia, Vol. 163, pp. 35-47, 2001. This discloses polymer matrices useful in biosensors that could be developed by immobilization of enzymes like glucose oxidase and aromatic redox-chromogenic structures at 6-deoxy-6-(4-aminophenyl)-aminocellulose. Also disclosed are p-toluenesulfonic acid esters of cellulose (tosylcelluloses) as intermediates, reacting with 1,4, phenylenediamine (PDA) to form “PDA cellulose.” PDA cellulose esters can then be formed into films onto which enzymes can be immobilized by glutardialdehyde reaction, diazo coupling, an ascorbic acid reaction, or other suitable means, as cited by Klemm and Einfeldt. No enzyme activity is lost within several days, according to the authors. The authors suggest biosensors using fiber optics to convey an optical signal. Redox-chromogenic properties were demonstrated by oxidative coupling reactions of phenols onto the PDA groups in the presence of H2O2 and peroxidase.

Another class of bioanalytical sensor has been developed that instead of using an enzyme to detect its substrate, senses the enzyme directly. This work is described by Michael R. Neuman in the publication, “Biomedical Sensors for Cost-Reducing Detection of Bacterial Vaginosis,” cect.egr.duke.edu/sensors.html, reporting work supported by NSF grant #9520526 and the Whitaker Foundation. Any suitable immunosensor and method of making the same can be used, including those of N. Trummer, N. Adányi, M. Varadi, I. Szendro in “Modification of the Surface of Integrated Optical Wave-Guide Sensors for Immunosensor Applications,” Fresenius Journal of Analytical Chemistry, Vol. 371, No. 1, August 2001, pp. 21-24, who disclose methods for attaching amino and epoxy groups to the surface of integrated optical wave-guide sensors for immunosensors. The SiO₂—TiO₂ surfaces were modified by use of the trifunctional silane reagents.

Lateral flow or immunochromatographic technology in any suitable form can be used in the biosensors as well. For example, Quidel (San Diego, Calif.) offers a variety of lateral flow devices that can be used in the present invention, including the QuickVue H.pylori gII test, which is a lateral-flow immuno-chromatographic assay intended for rapid detection of IgG antibodies specific to Helicobacter pylori in human serum, plasma or whole blood.

Biosensors can also function based on other scientific principles suitable for detection of analytes, including surface plasmon resonance (SPR), phase fluorescence, chemiluminescence, protein nucleic acid (PNA) analysis, baculovirus expression vector systems (BEVS), phage display, and the like. Examples of sensors incorporating such principles can be found in many sources, including the products of HTS Biosystems, such as their Proteomatrix™ Solution for proteomics. Basic information is provided at http://www.htsbiosystems.com/technology/spr.html. For example, HTS Biosystems' FLEX CHIP™ Kinetic Analysis System is based on grating-coupled SPR technology wherein measurements are made of optical properties of a thin film in close to a noble metal surface (e.g., gold or silver). Changes in molecular composition (e.g., when a target binds to a surface-bound capture probe) cause changes in the surface optical properties that are proportional to the amount of binding that occurs. The manufacturers state that this technology can be considered, in a way, to allow monitoring of surface-binding events in real time without the use of reporter labels. Grating-coupled SPR-based disposable biosensor chip can be made employing the technology currently used in producing digital video disc (DVD) media. An optical grating on a plastic base is produced. Amperometric immunosensors can also be used, such as those being developed at the Paul Scherrer Institute of Villigen, Switzerland, as described at Imn.web.psi.ch/molnano/immuno.htm. Biorecognition, the binding of antibodies to an antigen, for example, results in an electrical signal at an electrode. Antibodies are labeled with microperoxidase for generation of an electrochemical signal via electrocatalytic reduction of hydrogen peroxide. One application includes detection of antibiotics in milk, as described at Imn.web.psi.ch/molnano/penisens.htm and in Swiss Pat. Appl. No. 1764/99 (1999), by A. Grubelnik, C. Padeste and L. Tiefenauer.

Many forms of electrodes can be incorporated in the biosensors of value in the present invention. The electrodes can be created with photolithography, printing technologies such as ink-jet or screen printing, mechanical assembly, any technique suitable in the production of semiconductor chips, and the like. An example of screen-printed sensor is found in the work of A. J. Killard, et al. of Dublin City University, “A Screen-printed Immunosensor Based on Polyaniline,” described at www.mcmaster.ca/inabis98/newtech/killard0115/ and www.mcmaster.ca/inabis98/newtech/killard0115/two.html. Chips in biosensors can also include optical devices. For example, Motorola has developed a silicon chip integrated with a photon chip in which light-emitting gallium arsenide is bonded with strontium titanate to silicon (see Bill Scanlon, “Motorola Solves 30-Year Optical-Silicon Chip Puzzle,” Interactive Week, Sep. 10, 2001, p.18). Similar technology is being applied to bond light-emitting indium phosphide to silicon. Both approaches can be adapted for biosensors in which a chip generates and measures an optical signal that interacts with a medium to detect an analyte. Chips can also include light emitting diodes, diode lasers, or other light-emitting devices for biological sensing, as described, for example, in S. Dorato and A. Ongstad, “Mid-Infrared Semiconductor Laser Materials Engineering,” AFRL Technology Horizons, Vol. 2, No. 3, September 2001, pp. 14-15. Semiconductor lasers can generate beams in the near-IR spectral region (700-1000 nanometers). Bluegreen light can also be generated by semiconductor lasers, such as those based on III-V gallium nitrogen and II-VI zinc-sulfur compounds, which emit radiation in the range of 490 to 55 nanometers. Long wavelength diodes can also be used, with infrared radiation in the range of 2000 to 12,000 nanometers. Mid-IR devices, including tunable mid-IR semiconductor lasers, can also be used, as well as quantum-well lasers (e.g., a “W-laser”) and antimonide lasers.

Numerous biosensor chips can be used in the present invention, including those providing miniaturized, microfluidic assay chemistries. Exemplary devices are described in the article “Biochips” in Nature Biotechnology, Vol. 16, 1998, pp. 981-983, which also describes several examples of protein biochips, particularly the Affymetrix GeneChips. The p53 GeneChip, designed to detect single nucleotide polymorphisms of the p53 tumor-suppressor gene; the HIV GeneChip, is designed to detect mutations in the HIV-1 protease and also the virus's reverse transcriptase genes; and the P450 GeneChip focuses on mutations of key liver enzymes that metabolize drugs. Affymetrix has additional GeneChips in development, including biochips for detecting the breast cancer gene, BRCA1, as well as identifying bacterial pathogens. Other examples of biochips used to detect gene mutations include the HyGnostics modules made by Hyseq. Examples of biochips designed for gene expression profile analysis include Affymetrix's standardized GeneChips for a variety of human, murine, and yeast genes, as well as several custom designs for particular strategic collaborators; and Hyseq's HyX Gene Discovery Modules for genes from tissues of the cardiovascular and central nervous systems, or from tissues exposed to infectious diseases.

A wide variety of biosensor chips are provided by Biacore International AB (Uppsala, Sweden). Products are described at www.biacore.com/products/chips_all.shtml. In an example disclosed in the document at www.biacore.com/company/pdf/poster_ahm_use.pdf, a Biacore 3000 sensor was used to track the interaction of two enantiomers of a drug with human albumin. From this one can infer that real-time monitoring can be done of the interaction of a pharmaceutical agent with blood to assess the effectiveness of the drug. For example, a drug can be administered to the patient and a biosensor can then track the state of the drug in the blood to better guide application of the drug to the patient.

Another example is Caliper's LabChip, which uses microfluidics technology to manipulate minute volumes of liquids on chips. Applications include chip-based PCR as well as high-throughput screening assays based on the binding of drug leads with suitable drug targets.

In addition to suitable DNA and RNA-based chips, protein chips are being developed with increasing frequency. For example, a recent report describes the development of a quantitative immunoassay for prostate-specific membrane antigen (PSMA) based on a protein chip and surface-enhanced laser desorption/ionization mass spectrometry technology. Some protein biochips employ surface plasmon resonance (SPR). V. Regnault, et al. in British Journal of Haematology, Vol. 109, 2000, pp. 187-194 disclose the use of SPR to detect the interaction between autoantibodies and 2-glycoprotein I (a2GPI) immobilized on protein sensor chips, an interaction correlated with lupus. SPR enabled the interaction to be detected at a very low density of protein immobilization on the chip.

Microcantilevers and quartz crystals can serve as sensing elements for the detection of particular analytes, as described by C. Henry, “Biosensors Detect Antigens, Viruses,” Chemical and Engineering News, Vol. 79, No. 37, Sep. 10, 2001, p. 13. For example, G. Wu et al. in “Bioassay of Prostate-Specific Antigen (PSA) Using Microcantilevers,” Nature Biotechnology, Vol. 19, No. 9, September 2001, pp. 856-60, describe a sensitive microdevice employing microcantilevers that detects the presence of prostrate-specific antigen, a marker for early detection of prostrate cancer and for monitoring its progression. PSA antibodies are attached to a gold-coated silicon nitride microcantilever. Fluid passing over the device brings PSA, which binds to the antibodies, causing a change in the deflection of the microcantilever that can be measured by a laser. Levels of 0.2 ng/ml were detectable, even in a background of unrelated human serum proteins. The threshold for cancer detection of 4 ng/ml. Arrays of microcantilevers are possible, and could be employed to detect a plurality of analytes.

Quartz crystal microbalances (QCMs) have been used to detect viruses that bind to antibodies on the surface of the quartz, as described by M. A. Cooper, “Direct and Sensitive Detection of a Human Virus by Rupture Event Scanning,” Nature Biotechnology, Vol. 19, No. 9, September 2001, pp. 833-37. As the quartz crystal is oscillated an increasing frequencies in the presence of an alternating electrical field, a critical frequency is reached where the virus-antibody bond is ruptured. The quartz crystal, acting like an acoustic device, converts the acoustic emission from the bond rupture to an electrical signal. Proteins that are less strongly attached to the crystal are shaken off early during oscillation, allowing the device to distinguish between specific and non-specific adsorption.

A particularly sensitive class of microsensors includes acoustic sensors, such as those using surface acoustic wave (SAW), bulk acoustic wave (BAW), and acoustic plate modes (APM). Selectivity is typically achieved by coating a thin polymeric or metallic film on the sensing surface of the piezoelectric crystal. The polymer may be organic, inorganic or organometallic. Acoustic wave chemical sensors and biosensors thus consist of a piezoelectric crystal device and a chemical system attached to the crystal surface. The chemical system consists of the polymeric coating and/or chemoreceptors attached to the coating. The chemical system is used as a molecular recognition element and has the ability to selectively bind molecules and gas particles. While the physics of the detection process is very complex, the principle of operation of acoustic wave device sensor is quite simple and the results are reliable. An acoustic wave confined to the surface (SAW) or bulk (BAW) of a piezoelectric substrate material is generated and allowed to propagate. Any matter that happens to be present on the crystal surface will perturb that surface in such a way as to alter the properties of the wave (i.e. velocity or frequency, amplitude or attenuation). The measurement of changes in the wave characteristics is a sensitive indicator of the properties of the material present on the surface of the device. In general, it is well known that both mechanical and electrical perturbations of the surface affect the propagating acoustic waves and result in sensing. Such perturbations result from the absorption or diffusion of gas into the film; molecule selectivity, migration or binding; and formation of complexes within the film.

A useful example of a piezoelectric sensor is given in U.S. Pat. No. 5,852,229, “Piezoelectric Resonator Chemical Sensing Device,” issued Dec. 22, 1998 to Josse and Everhart, incorporated herein by reference. Josse and Everhart disclose a sensor including a piezoelectric resonator having a first side with an electroded region and a second opposing side having an electroded region that is different in size and/or shape of the first electrode. The piezoelectric resonator of the present invention is capable of measuring more than one parameter thereby providing a multi-information-sensing device. The present invention also includes an apparatus and method for detecting and measuring an analyte in a medium that utilizes the piezoelectric resonator sensor of the present invention.

### (1) Diffraction-Based Technologies

A variety of diffraction-based technologies can be employed in making low-cost biosensors. For example, U.S. Pat. No. 5,922,550, “Biosensing Devices Which Produce Diffraction Images,” issued Jul. 13, 1999 to Everhart et al., incorporated herein by reference, discloses a disposable biosensor which can be used to detect many analytes. The device includes a metalized film upon which is printed a specific predetermined pattern of analyte-specific receptors. Upon attachment of a target analyte, which is capable of scattering light, to select areas of the plastic film upon which the receptor is printed, diffraction of transmitted and/or reflected light occurs via the physical dimensions and defined, precise placement of the analyte. A diffraction image is produced which can be easily seen with the eye or, optionally, with a sensing device. By “diffraction” it is meant the phenomenon, observed when waves are obstructed by obstacles, of the disturbance spreading beyond the limits of the geometrical shadow of the object. The effect is marked when the size of the object is of the same order as the wavelength of the waves. In the U.S. Pat. No. 5,922,550 patent, the obstacles are analytes and the waves are light waves.

Everhart et al. in U.S. Pat. No. 5,922,550 employ methods of contact printing of patterned, self-assembling monolayers of alkanethiolates, carboxylic acids, hydroxamic acids, and phosphonic acids on metalized thermoplastic films, the compositions produced thereby, and the use of these compositions. The self-assembling monolayers have receptive materials bound thereto. The receptive materials are specific for a particular analyte or class of analytes depending upon the receptor used.

Patterned self-assembling monolayers allow for the controlled placement of analytes thereon via the patterns of analyte-specific receptors. The biosensing devices of the present invention produced thereby are used by first exposing the biosensing device to a medium that contains the analyte of choice and then, after an appropriate incubation period, transmitting a light, such as a laser, through the film. If the analyte is present in the medium and is bound to the receptors on the patterned self-assembling monolayer, the light is diffracted in such a way as to produce a visible image. In other words, the patterned self-assembling monolayers with the analyte bound thereto can produce optical diffraction patterns that differ depending on the reaction of the receptors on the self-assembling monolayer with the analyte of interest. The light can be in the visible spectrum, and be either reflected from the film, or transmitted through it, and the analyte can be any compound or particle reacting with the self-assembling monolayer. The light can be a white light or monochromatic electromagnetic radiation in the visible region. The present invention also provides a flexible support for a self-assembling monolayer on gold or other suitable metal or metal alloy.

Everhart et al. in U.S. Pat. No. 5,922,550 further disclose a support for a self-assembling monolayer on gold or other suitable material which does not require an adhesion promoter for the formation of a well-ordered self-assembling monolayer. They also disclose a support for a self-assembling monolayer on gold or other material that is suitable for continuous printing, rather than batch fabrication, allowing the device to be mass produced. Their biosensor can be produced as a single test for detecting an analyte or can be formatted as a multiple test device, and can be used to detect contamination in garments, such as diapers, and to detect contamination by microorganisms.

Other diffraction-based biosensors are disclosed in the following patents, all of which are incorporated herein by reference:

U.S. Pat. No. 6,060,256, “Optical Diffraction Biosensor,” issued May 9, 2000 to Everhart et al., which discloses a metalized film upon which is printed a pattern of analyte-specific receptors. Upon attachment of a target analyte to select areas of the plastic film upon which the receptor is printed, diffraction of transmitted and/or reflected light occurs to produce a diffraction image that can be easily seen with the eye or with a sensing device.

U.S. Pat. No. 6,221,579, “Patterned Binding of Functionalized Microspheres for Optical Diffraction-Based Biosensors,” issued Apr. 24, 2001 to D. Everhart, R. Kaylor, and K. McGrath, discloses additional diffraction-based techniques for biosensors, including an embodiment in the form of a dip stick. In one aspect, the invention includes a diffraction enhancing element, such as functionalized microspheres, which are modified such that they are capable of binding with a target analyte. Additionally, the system includes a polymer film, which may include a metal coating, upon which is printed a specific, predetermined pattern of analyte-specific receptors. Upon attachment of a target analyte to select areas of the polymer film, either directly or with the diffraction-enhancing element, diffraction of transmitted and/or reflected light occurs via the physical dimensions and defined, precise placement of the analyte. A diffraction image is produced which can be easily seen with the eye or, optionally, with a sensing device.

WO 00/36416, “Patterned Deposition of Antibody Binding Proteins for Optical Diffraction-Based Biosensors,” published Jun. 22, 2000, by K. McGrath, R. Kaylor, and D. Everhart, which discloses a biosensor including: a polymer film; and an antibody-binding protein layer printed in a pattern onto the polymer film wherein the antibody-binding protein layer has an antibody thereon that is specific for an analyte.

U.S. Pat. No. 6,020,047, “Polymer Films Having a Printed Self-Assembling Monolayer,” issued to D. S. Everhart, Feb. 1, 2000, and U.S. Pat. No. 6,048,623, “Method of Contact Printing on Gold Coated Films,” issued Apr. 11, 2000 to Everhart et al., which disclose sensors formed by applying patterned, self-assembling monolayers of alkanethiolates, carboxylic acids, hydroxamic acids, and phosphonic acids on suitable substrates. Patterned self-assembling monolayers allow for the controlled placement of materials thereon which contain a chemically reactive, indicator functionality. The optical sensing devices produced thereby when the film is exposed to an analyte and light, can produce optical diffraction patterns which differ depending on the reaction of the self-assembling monolayer with the analyte of interest.

U.S. Pat. No. 6,180,288, “Gel Sensors and Method of Use Thereof,” issued Jan. 30, 2001 to Everhart et al., which discloses an optically diffracting sensing device whose diffraction pattern changes upon exposure to an analyte. The device includes one or more gels coated onto patterned, self-assembling monolayers of alkanethiolates, carboxylic acids, hydroxamic acids, and phosphonic acids printed onto a variety of substrates, including glass, silicon, aluminum oxide, and thermoplastic films metallized with gold, or with an alloy such as nickel/gold.

### (2) I-Stat Biosensors

Useful biosensors for the present invention are exemplified by several of the products of i-STAT Corporation (East Windsor, N.J.). The I-STAT System uses micro-fabricated thin film electrodes as electrochemical sensors whose signals can be measured and quantified with the I-STAT Portable Clinical Analyzer's amperometric, pontentiometric, or conductometric circuits. Solution for calibrating the electrodes is provided in a foil pouch within the measurement cartridge. During measurement of either the calibrating solution or a blood sample, the fluid being measured flows over a sensor array for measurement. Measurements are made by ion-selective electrode potentiometry for sodium, potassium, chloride, ionized calcium, pH, and pCO₂. Also measured are urea (after hydrolysis to ammonium ions by urease), glucose (amperometric measurement of hydrogen peroxide produce from glucose by the enzyme glocose oxidase); pO₂ (using an electrode similar to a conventional Clark electrode, with oxygen diffusing from the blood through a gas permeable membrane into an internal electrolyte solution, where it is reduced at a cathode to generate a current), and hematocrit (measured conductometrically). Additional results can be calculated for HCO₃ (bicarbonate), TCO₂ (total carbon dioxide, the sum of the carbonic acid and bicarbonate levels), BE (base excess), sO₂ (saturated oxygen), anion gap and hemoglobin.

Several biosensor technologies are disclosed in a U.S. patent assigned to I-Stat Corp., U.S. Pat. No. 5,063,081, “Method of Manufacturing a Plurality of Uniform Microfabricated Sensing Devices Having an Immobilized Ligand Receptor,” issued Nov. 5, 1991 to Cozzette et al., incorporated herein by reference. Disclosed therein are wholly microfabricated biosensors having a plurality of thin films and related structures over a planar wafer. The sensors employ biologically active macromolecules and other reagents necessary for the conversion of selected analyte molecules to more readily detectable species, typically using electrochemical assay procedures for determining the presence and/or concentration of biological species (analytes) of interest. A substrate is used that does not undergo detectable electrochemical oxidation or reduction but which undergoes a reaction with a substrate converter producing changes in the concentration of electroactive species. These changes are measured and related proportionately to the concentration of the analyte of interest. The substrate converter can be an enzyme that hydrolyzes the substrate. This hydrolyzed substrate can then undergo reactions which produce changes in the concentration of electroactive species (e.g., dioxygen and hydrogen peroxide) which are electrochemically detected with the biosensor, e.g., a ligand/ligand receptor-based (LLRbased) biosensor in this instance. Both sandwich and competitive assays can be used.

In one immunoassay system disclosed by Cozette et al., a biosensor includes a catalytic electrode and optional reference electrode (base sensor), an adhesion promoter layer overlaid on the biosensor, and a bioactive layer that is immobilized on the adhesion promoter layer, which bioactive layer is a receptor (first member) of the immunological analyte of interest. The wholly microfabricated biosensor includes a wafer on which a first structure including a suitable base sensor is established. Additional structures are then established over the resulting base sensor, which additional structures include a semipermeable solid film or permselective layer capable of acting as a barrier against interfering chemical species while allowing the transport of smaller detectable chemical moieties of interest. These detectable chemical moieties are typically electroactive molecules and may include low molecular weight ionic species. The semipermeable solid film may further include compounds or molecules that may serve to sensitize the base sensor to a preselected ionic species (e.g., ammonium ion). Furthermore, such permselective layers may also function as adhesion promoters by which the preselected ligand receptor may be immobilized to the wholly microfabricated LLR-based biosensor embodiment of the present invention. The support matrices described by Cozette at al. can possess or support the physical and chemical features necessary for converting the particular analytes in a given analytical sample into detectable and/or quantifiable species. Techniques are disclosed for localizing or patterning said matrices on certain desired areas of the wholly microfabricated biosensor which allow for the optimum control over dimensional features of the biolayers as well as the versatility to accommodate a wide range of bioactive molecules. Additionally, the overlaid structures can be provided for the attenuation of the transport of selected analyte species that are present in high concentrations in the sample. Such analyte attenuation (AA) layers allow for a linear sensor response over a wider range of analyte concentrations than would be observed in the absence of an AA layer. Furthermore, the overlaid AA layer, which can be derived from a siloxane/nonsiloxane copolymer, is capable of excluding very large molecules or other contaminating constituents of the sample whose direct contact with the underlying structures would result in interference with or fouling and an eventual reduction in the reliability of the biosensor. If the AA layer is of the appropriate structure and composition, it may also function as a gas permeable membrane. In certain embodiments, such a gas permeable membrane can allow only very small molecules to pass through. The gas permeable membrane also insulates the immediate environment of the electrode portion of the biosensor from external fluid turbulence. Thus, the measurements performed by the preferred LLR-based sensor can be rendered substantially free of flow dependence.

Apart from the AA layer mentioned above, a semipermeable solid film that is able to function as a molecular weight-sensitive transmissive film is among the layers. Depending upon the composition and final thickness of this semipermeable solid film, also referred to as a permselective layer, molecules having molecular weights above a given threshold can be effectively excluded from entering and diffusing through such a film. As a general illustration of the function and utility of this permselective layer, molecules having a molecular weight of about 120 or above are effectively blocked by a solid film having a thickness of about 5 to about 10 nm. Varying degrees of control over the size of the molecules excluded and the rates of transport of smaller molecules which are able to diffuse through the solid film can be obtained with solid films having a thickness in the range of about 2 to about 50 nm. With certain types of materials, these permselective layers may be as thin as 1 nm or may be as thick as 100 nm. This film may be established on the substrate wafer or any planar analyte-sensing device in a number of ways but most conveniently as an initial liquid film, including a silane compound mixed with a suitable solvent, which is spin-coated across the wafer. If desired, the permselective layer may be formed at specific preselected areas of the device by means of photolithographic processing techniques. Techniques such as “lift-off” and use of a photoresist cap in combination with a plasma-etching or, alternatively, a wet-etching step may thus be employed to define the location and configuration of the semipermeable solid film. The initial liquid silane mixture, like many other liquid mixtures of use in the present invention, can also be microdispensed at multiple preselected areas of the sensing device. Such microdispensing of fluid media may be performed automatically and in uniform predetermined quantities by a computer-controlled syringe interfaced with the controlled movements of a vacuum chuck holding the substrate wafer. Such microdispensing techniques are consistent with a microfabrication method and are discussed in further detail in Cozette et al. Thus, in an amperometric electrochemical sensing device, interfering electroactive species having a molecular weight above a desired threshold (e.g., above 120) may effectively be excluded from interacting with the catalytic electrode surface by employing a permselective layer that still allows lower molecular weight electroactive species, like dioxygen and hydrogen peroxide, to undergo a redox reaction with the underlying electrode surface.

### (3) Hormone and Pregnancy-Related Sensors

Biosensors may be used to assist in hormone therapy used, for example, to prevent or treat osteoporosis or other problems. The balance of hormones applied may need to change over time, and the correct balance may be inferred from biosensors responsive to hormone levels in the blood or other indicators such as bone mineral density or other chemical analytes. In response to a biosensor signal, for example, a physician may modify the hormone balance provided to a patient. The adjusted medication may be ordered electronically from a pharmacy, and the medication may be delivered to the subject or provided by a nurse or other caregiver.

Direct detection of enzymes in biosensors can be useful in many aspects of health care, particularly for feminine care and pregnancy monitoring. The enzyme-detection sensors referred to in the above-mentioned work of Neuman can be of particular value. Neuman observes that since diamineoxidase is found in amniotic fluid, this type of sensor may also be useful in detecting premature rupture of membranes with leakage of fluid when conventionally used techniques provide equivocal results. A preliminary design for an intervaginal probe has been reduced to practice and investigators are designing a probe that will contain 4 pH sensors for mapping intervaginal pH. Such probes can be used within the scope of the present invention.

Such devices can employ both a potentiometric pH sensor and an amperometric diamine sensor to aid in vivo diagnosis of bacterial vaginosis (BV). Techniques are known to make single-site diamine sensors on a flat-form, self-contained sensor substrate that has been batch-fabricated on a flexible polyimide layer.

For pregnancy monitors to predict a possible premature delivery, several options are available. Recent work has shown that electrodes can detect early contractions of the uterus days or weeks in advance of delivery to signal the onset of labor (see New Scientist, Mar. 2, 2001). Thus, electrodes placed on an expecting mother could be used to monitor contractions well before the onset of delivery.

A pad that can be worn by a woman to detect premature delivery is disclosed in WO 00/04822 or EP 1,098,590.

Biochemical means can also detect the onset of delivery in advance. George C. Lu et al. in “Vaginal Fetal Fibronectin Levels and Spontaneous Preterm Birth in Symptomatic Women,” Obstetrics and Gynecology, Vol. 97, No. 2, February 2001, pp. 225-228, incorporated herein by reference, establish that detection of fibronectin in the vagina is an indicator of preterm birth. Fibronectin is a protein produced by the chorioamniotic membranes and apparently serves as a biological glue that maintains the integrity of structures in the womb. Lu et al. review evidence that disruption of those structures (the chorionicdecidual interface) precedes preterm labor and causes the release of fetal fibronectin into the cervicovaginal fluid. Several technologies exist for detection of fibronectin that could be adapted for a disposable home-use biosensor. Those of Adeza Corp., for example, can be used.

Other analytes related to premature rupture of the amniotic membrane include hCG, IGFBP-1, alpha FP, and diamine oxidase. Further, monitoring of nitrate and nitrite levels in the body can be correlated with premature delivery. Sensors useful for these analytes are described hereafter. Prolactin can also be monitored as an indicator of premature labor. For prolonged pregnancy, fetal fibronectin biosensors can again be useful.

U.S. Pat. No. 6,149,590, incorporated herein by reference, discloses the use of pH sensitive paper, including nitrazine paper, that is liquid permeable, for identification of premature membrane rupture in pregnancy. Amniotic fluid changes the color of the paper. This can be incorporated into a sanitary napkin.

Estriol, alpha fetoprotein, human chorionic gonadotropin (hCG), and inhibin-A are other analytes of value in pregnancy monitoring.

Antiphospholipid Syndrome (APS) is a health problem affecting many women. The presence of antiphospholipid antibodies in the body is often associated with pregnancy loss, and APS also can cause thrombosis in veins or arteries of the woman, as discussed by N. B. Chandramouli and G. M. Rodgers in “Management of Thrombosis in Women with Antiphospholipid Syndrome,” Clinical Obstetrics and Gynecology, Vol. 44, No. 1, 2001, pp. 36-47. W. Geis and D. W. Branch discuss antiphospholipid antibodies and their relationship to pregnancy loss in “Obstetric Implications of Antiphospholipid Antibodies: Pregnancy Loss and Other Complications,” Clinical Obstetrics and Gynecology, Vol. 44, No. 1, 2001, pp. 2-10.

APS can be detected by immunoassay tests or other tests, as described by S. S. Pierangeli, A. E. Gharavi and E. N. Harris in “Testing for Antiphospholipid Antibodies: Problems and Solutions,” Clinical Obstetrics and Gynecology, Vol. 44, No. 1, 2001, pp. 48-57. It is often desirable to verify the presence of the syndrome by using two different tests. Immunologic assays can be used that directly detect antiphospholipid antibodies or to detect LA or related proteins. Enzyme-Linked immunosorbent Assay (ELISA) systems can also be used.

Another useful marker may be human chorionic gonadotropin (hCG), which is usually used to determine whether a woman is pregnant. In addition, however, marker can continue to be monitored as an indicator of the health of the fetus. TPS can also be monitored.

Noninvasive optical sensors can also be used to pass light through the abdomen of the mother and reach the fetus, allowing measurement of blood oxygen levels with pulse oximetry, as described in N. D. Rowell, “Light Could Help Doctors Draw Less Blood,” Photonics Spectra, September 2001, pp. 68-72. See also A. Zourabian et al., “Trans-abdominal Monitoring of Fetal Arterial Blood Oxygenation Using Pulse Oximetry,” Journal of Biomedical Optics, October 2000, pp. 391-405.

Biosensors according to the present invention can be used for monitoring of folic acid in pregnant women or in women planning to become pregnant. A particular challenge exists for many of those who have used oral contraceptives, where folic acid levels are often low and body reserves have been depleted. It has been recommended that these women wait for several months to regain the folic acid levels needed for a healthy pregnancy. Monitoring of folic acid levels in the body can be helpful in preparing for a healthy pregnancy and maintaining health of the mother and fetus during pregnancy.

In addition to monitoring folic acid in the body, in some cases it may be desired to monitor intake of folic acid with suitable sensors. Biacore sensors, among others, can be used for this application. T. A. Grace et al. of Biacore describe the use of a surface plasmon resonance sensor (Biacore Q sensor system) for folic acid determination in the paper, “The Determination of Water-Soluble Vitamins in a Variety of Matrices by Biacoreq Assay Kits,” Institute of Food Technologists Annual Meeting, June 2001, New Orleans (abstract available at ift.confex.com/ift/2001/tech program/paper_{—}9594. htm—see also www.biacore.com/customer/pdf/vol2no2/22p22.pdf). Samples of foodstuffs can be blended, ground, and optionally centrifuged in the preparation of extracts suitable for direct measurement of folic acid levels with sensors. Another example of a Biacore biosensor system for folic acid determination is described by M. Boström-Caselunghe and J. Lindeberg, “Biosensor-Based Determination of Folic Acid in Fortified Food,” Food Chemistry, Vol. 70, 2000, pp. 523-32.

A marker of use in predicting ectopic pregnancy is “smhc Myosin,” as well as serum progesterone.

Pre-eclampsia (formerly known as “toxaemia”), a hypertensive disorder of pregnancy associated with proteinuria and pathologic edema, may be tracked by monitoring protein in the urine or other factors.

Numerous home test devices exist for detecting pregnancy or the onset of ovulation, any of which can be adapted for the present invention. Basal temperature measurements and urine LH (luteinizing hormone) kits represent two common technologies. Monitoring Follicle Stimulating Hormone with biosensors in absorbent articles to track the onset of ovulation is suggested in the following U.S. patent applications: Ser. No. 09/299,399, filed Apr. 26, 1999; Ser. No. 09/517,441, filed Mar. 2, 2000; and Ser. No. 09/517,481, filed Mar. 2, 2000; each of which was previously incorporated by reference.

Biosensors for fertility monitoring and the detection of ovulation include those of Thermo BioStar, Inc. (Boulder, Colo.); the TFS estradiol metabolite BioSensor of ThreeFold Systems, Inc. (Ann Arbor, Mich.); the OvuSense biosensor of Conception Technology Inc. (Longmont, Colo.); and Pheromone Sciences Corp. (Toronto, Canada), whose PSC Fertility Monitor is worn like a watch and uses non-invasive measurement of ions on the skin. The PSC Fertility Monitor incorporates an interactive microprocessor combined with a biosensor enabling it to take up to 12 daily measurements from the skin surface and to evaluate the data in order to predict the status of the user as being not-fertile, fertile, or ovulating. Results can be viewed at any time on the LCD screen of the device or as a computer-generated graphical printout for medical professionals. Further examples include U.S. Pat. Nos. 6,234,974 and 5,656,503 assigned to Unilever, and WO 99/10742 assigned to Fertility Acoustics.

### (4) Sensors for Vaginosis

Biosensors can also be used for the detection of yeast vaginitis or bacterial vaginitis. Sensors can respond to pH changes associated with such conditions, and can also detect another physical or chemical condition, such as the presence of a diamine, for increased accuracy. Exemplary biosensors include those developed by Michael R. Neuman, as described in the publication, “Biomedical Sensors for Cost-Reducing Detection of Bacterial Vaginosis,” available on the Internet at cect.egr.duke.edu/sensors.html, reporting work supported by NSF grant #9520526 and the Whitaker Foundation. Such sensors are based on thin-films on polyimide microstructures. These sensors can also be used to detect pH changes associated with premature rupture of amniotic membranes and the release of amniotic fluid. In one embodiment described therein, the enzyme layer was immobilized on the working electrode surface by crosslinking putrescine oxidase (PUO) with bovine serum albumin using glutaraldehyde. The three-electrode sensor prepared was sensitive to putrescine.

A pH-based method for distinguishing between yeast infections and other secretion-causing conditions employing a color-changing sensor in an absorbent article is disclosed in U.S. Pat. No. 5,823,953, issued Oct. 20, 1998 to Roskin et al., incorporated herein by reference. The sensor and/or article of Roskin can be used within the scope of the present invention.

Bacterial pathogens can be tracked by monitoring vaginal pH (e.g., using biosensors from Litmus Concepts, Inc. of Santa Clara, Calif.), ECA, or alpha antigen, or by other suitable techniques. Lactoferrin is another biological analyte related to vaginosis that can be monitored with biosensors. Detection of proline aminopeptidase or other amines can be achieved using biosensors from Litmus Concepts, Inc. and applied to vaginosis tracking.

Volatile Organic Compounds (VOCs) produced by the bacteria and yeast associated with vaginosis can also be detected with biosensors to detect vaginosis and monitor healing. Vaginosis is usually due to a change in the balance among different types of bacteria in the vagina. Instead of the normal predominance of Lactobacillus, increased numbers of organisms such as Gardnerella vaginalis, Bacteroides, Candida, Mobiluncus, and Mycoplasma hominis are found in the vagina in women with vaginosis.

One of the most common causes of vaginitis in women is Candida albicans. Almost every woman experiences a yeast infection at some point in her life and many women are plagued by recurring episodes of vaginal yeast infections. There are several different strains of Candida which are implicated with vaginosis. The most common symptoms of this type of vaginosis are a thick white discharge and intense itching and sometimes burning, both inside and outside the vagina. There may at times be an odor, but this is not usually considered the primary symptom. In one embodiment, the biosensor monitors odors specifically produced by C. albicans as a marker for vaginitis.

The bacteria Gardnerella is another common cause of yeast infections. Again, it is possible to monitor odors, enzymes, or other compounds specifically produced by Gardnerella as a predominant marker for association with vaginitis. Another vaginal infection that is less common is Trichomonas. This protozoan infection is usually sexually transmitted. Again, it is possible to monitor odors specifically produced by Trichomoniasis as a marker for vaginitis.

Traditionally, diagnoses for vaginosis are made microscopically. A vaginal infection can be precisely identified by a three-minute, three-step testing procedure on a single sample of vaginal discharge. The testing requires pH paper, potassium hydroxide, saline solution, and a microscope. The draw back of this procedure is that it requires trained medical professionals to complete the diagnosis. A rapid simple measure available to the consumer would allow for more timely treatment of vaginosis and a benefit to public health.

Anaerobic and facultative bacteria that normally live on and in the skin as well as on and in mucus membranes commonly cause odors. Anaerobic growth of these organisms requires an organic compound as a terminal electron (or hydrogen) acceptor. Simple organic end products are formed from the anaerobic metabolism of carbohydrates and/or some other compound. The simple organic end products formed from this incomplete biologic oxidation process also serve as final electron and hydrogen acceptors. Upon reduction, these organic end products are secreted by the bacterium as waste metabolites. Many of these compounds are VOCs. Thus, a biosensor can monitor these VOCs allowing for the identification of the type of microbe infecting the vagina and associated vaginosis. It has been established that the type and pattern of VOCs produced by microbes can be associated with specific classification.

Micro-arrays can be employed to detect the volatiles. Arrays of electronic sensors (e.g., electronic nose technology), capable of detecting and differentiating complex mixtures of volatile compounds, have been utilized to differentiate aromas of food and related materials. Electronic nose technology can contain an array of sensors, using a variety of different sensor technologies. Conducting polymer sensors are the most common sensors, as exemplified by the devices of the University of Warwick (Coventry, England), Neotronics Scientific Ltd. (Bishops Stortford, England), AromaScan Inc. (Hollis, N.H.), and Cyrano Sciences, Inc. (Pasadena, Calif.). Oligomeric sensors are reportedly stable, durable, and easy to use, such as the devices studied at the University of Antwerp. Metal oxide sensors are inexpensive to produce and said to be simple to operate, exemplified by the diAGnose agricultural sensor of Texas A&M University and gas sensor chips from Hong Kong University of Science & Technology. Quartz microbalance technology has also been used to develop an indicator system that responds to a wide range of compounds, as demonstrated at Griffith University (Brisbane, QLD), and RST Rostock (Warnemunde, Germany). Electronic nose technology is also described by T. -Z. Wu, “A Piezoelectric Biosensor as an Olfactory Receptor for Odour Detection: Electronic Nose,” Biosensors and Bioelectronics, Vol. 14, 2000, pp. 9-18. Another sensor for detecting chemicals in the gas phase is the chemical sensor badge developed by Nicholas L. Abbott, a professor ,of chemical engineering at the University of Wisconsin, and Rahul R. Shah of 3M Corporation, as reported in the NASA Tech Briefs Sensors Newsletter of Sep. 19, 2001. These sensors do not require electrical power, and provide direct visual indications of the presence of a chemical. Designed using nanotechnology, they use microscopic liquid crystals attached by a few molecules of a chemically receptive substance to a thin film of gold. When the substance is exposed to chemicals, it bonds to the targeted chemical, and loosens its grip on the liquid crystal. The crystals take on a new orientation controlled by the texture of the gold surface, and the result is visible as a change in the sensor's brightness or color. The substrate can be a flexible polymeric material that is fastened to the outside of an article of clothing. Multiple sensors for multiple analytes could be used.

One useful multi-analyte sensor is disclosed by C. Hagleitner et al. in “Smart Single-Chip Gas Sensor Microsystem,” Nature, Vol. 414, 2001, pp. 293-96. They disclose a smart single-chip chemical microsensor system that incorporates three different transducers (mass-sensitive, capacitive, and calorimetric), all of which rely on sensitive polymeric layers to detect airborne volatile organic compounds. Full integration of the microelectronic and micromechanical components on one chip permits control and monitoring of the sensor functions, and enables on-chip signal amplification and conditioning that notably improves the overall sensor performance. The circuitry also includes analog-to-digital converters, and an on-chip interface to transmit the data to off-chip recording units. This technology may be applied to produce improved noses or other gas-phase sensors, which can also be used in cooperation with liquid-phase or other sensors to simultaneously examine a wide variety of analytes.

The applications of these arrays to detect VOCs produced by problem microbes require that the array be modified to detect the compounds specific to those organisms. Compounds that can be monitored include, without limitation, oxalacetic acid, pyruvic acid, malonic acid, lactic acid, formic acid, acetic acid, fumaric acid, caproic acid, dimethyl disulfide, ammonia, acetone, isovaleric acid, and triethylamine. The biosensor signal can include a stand-alone chip that is placed in a non-woven, coform, or cellulosic material such that the signal is either generated as a color change or electronic voltage.

### (5) Other Women's Health Issues

Biosensors can also be used to detect the onset of menopause and track a woman's health after menopause. Useful biological markers for these purposes include transferrin, serum ferritin, inhibins A and B (e.g., using technologies of DSL, Inc.), FSH, estradiol, inflammatory cells, MMPs, and reproductive hormones. Ferritin and hemoglobin can be tracked to assess iron status during menstruation. Nitrogen oxides can also be tracked to assess menstrual homeostasis. Bone resorption or osteoporosis can be related to monitored levels of CA-125, osteocalcin, C- or N-telopeptides from collagen (CTx or NTx, respectively), pyridinoline (PYD) and deoxypyridinoline (DYD), etc. Endometrial health can be related to desmin, CEA, PP10, P12, PP14, and PP15, while endometriosis can be monitored via CD23, perforin, Grannzyme B, CA-125, CA72-4, CA19-9, MMP-7, MMP-9, and TIMP.

Ovarian dysfunction can be related to measurements of anti-corpus leuteum antibodies, CA-125, estradiol, and testosterone. Cervical health can be related to mucous glycoconjugates, and alpha subunit hCG. Vaginal health can be tracked with serym amyloid-P componen, Nafarelin, and pH monitoring, in addition to other means previously discussed. Toxic shock can be detected with serum TS antibodies (e.g., using a biosensor associated with a tampon). PID and chronic pelvic pain may be related to CA-125 levels. The probability of egg implantation can be monitored through measurements of placental protein PP14, MMP, and IGFBP-3, while fertility and cycle monitoring can be tracked to some degree by measurements of circadian temperature, PP5, PP10, PP15, and hDP200.

Monitoring of MW antigen can be useful as an indicator of cervical dysplasia or bleeding.

Progesterone or hLH beta core fragments in urine can also be monitored for prediction of menopause.

### (6) Sexually-Transmitted Diseases (STDs)

STDs such as chlamydia or gonorrhea can be detected by analysis of components in urine with a DNA-based test using a benchtop system by Cepheid. STDs are another large category of diseases that could readily be monitored with biosensors in disposable absorbent articles, and tied to an integrated health care system.

### (7) Saliva-Based Tests

Biosensors for detecting analytes in saliva can be used. Examples include products of Salimetrics (State College, Pennsylvania), which provides a suite of salivary enzyme-immunoassay (EIA) kits for analytes such as cortisol (an indicator of stress), DHEA (dehydroepiandrosterone), testosterone, estradiol, progesterone, melatonin, cotinine, neopterin, and sIgA (secretory immunoglobulin A). The Male/Female Testosterone Profile test kit and the Post Menopausal Panel (for hormone detection) of are also a saliva-based system. Saliva-based fertility testing devices are also commercially available for predicting the time of ovulation, including the “Lady Fertility Tester” distributed by Med-Direct.com.

Related innovations have been developed by Dr. Douglas Granger at Pennsylvania State University, as described by D. A. Granger et al., “Salivary Testosterone Determination in Studies of Child Health and Development,” Hormones and Behavior, Vol. 35, 1999, pp. 18-27, which discloses techniques for measuring hormones in children's saliva. See also www.hhdev.psu.edu/news/hhdmag/fall% 201999/fluid.html, which provides an overview of Granger's work, describing applications such as cancer screening, HIV detection, hormone tracking (DHEA, progesterone, etc.), cortisol, and a variety of other analytes normally measured in the blood.

### (8) Test Strips

The lateral flow immunochromatographic tests produced by Chembio Diagnostic Systems, Inc. (see chembio.com/tech.html) are one example of biosensor systems within the scope of the present invention. These test materials are designed for qualitative detection of various analytes. Based on the differences in their operational procedures, these immunologic test devices fall into three general categories: (1) one-step, lateral flow devices that detect hCG, hLH, PSA, Hepatitis-B surface antigen, Troponin-I, etc.; (2) two-step lateral flow devices detect antibodies to H-pylori, Mycobacterium tuberculosis, Trypanosoma cruzi (Chagas), Borrelia burgdorferi (Lyme), etc. in whole blood, serum or plasma; (3) assays that require off-line extraction of antigen before their detection, including assays for Chlamydia, Strep-A, Rotavirus, etc. The extraction procedures are said to be simple, rapid and to require no additional equipment.

The Chembio test strips use colloidal gold conjugates. These colloidal gold conjugates are stored in dry mobile state in the devices. On coming into contact with biological samples, the colloidal gold conjugate quickly becomes re-suspended and binds to antigen or antibody in the sample and moves across the membrane through capillary migration. If the colloidal gold has captured the specific antigen or antibody then a second antibody or antigen, immobilized at the test zone, captures the colloidal gold-coupled immune complex. A pink/purple line appears in the test zone. The intensity of the line color may vary with the concentration of the antigen or antibody.

### (9) Implanted Biosensors

Biosensors that require surgical implantation of a component in the body can also be used. Examples include chemical sensors that continuously monitor an analyte such as a protein or blood component. Implantable biosensor components can also include biosensor chips with an internal power source for generating signal. An implanted component can also be free of electronic devices or power sources, but can yield a signal in response to applied radiation, such as optical or microwave radiation. One example includes the implantable silicon-based mirrors described in N. D. Rowell, “Light Could Help Doctors Draw Less Blood,” Photonics Spectra, September 2001, pp. 68-72. Such implantable mirrors have been developed by pSiMedica (Malvern, UK), intended to improve noninvasive optical measurements of tissue or blood for detection of glucose levels, oxygen levels, and cancer detection. The mirrors can be 5 mm×0.5 mm, for example, and include alternating layers of highly porous and less porous silicon. The different refractive index of the layers reflects beams of light at the interface with interference occurring that affects that wavelength of the reflected beam. The reflected wavelength can be controlled by the thicknesses of the alternating layers. The mirrors can reflect near-infrared light that is not scattered by the tissue. The pores in the silicon can be filled with chemicals that bind to specific markers. Cancer markers or other components can bind and accumulate in the pores, changing the reflectivity of the mirror. An infrared beam shone onto a mirror from outside the body can then be reflected from the mirror, and the measured reflectivity can indicate the presence of markers in the pores.

The mirrors can break down to harmless silicic acid in the body, and theoretically can be adjusted to break down over a period of hours to years. Further information is provided in L. T. Canham et al., “Derivatized Porous Silicon Mirrors: Implantable Optical Components with Slow Resorbability,” Physica Status Solidi, November 2000, pp. 521-25.

### (10) Other Systems

Any of the following biosensor systems and concepts can also be employed in the present invention. Each of the patents mentioned below and elsewhere in this document is incorporated by reference.

The sensor of U.S. Pat. No. 6,231,733, “Immobilized Carbohydrate Biosensor,” issued May 15, 2001 to Nilsson et al., which discloses a biosensor in which a carbohydrate or a derivative of a carbohydrate is used to generate a detectable signal by way of the specific binding to a protein, a virus or a cell.

The sensor chips of JP 2001/056340-A by the Japanese Agency of Industrial Science and Technology, Aug. 18, 1998. These sensor chips are for detecting a trace substance such as hormones or enzymes in the blood. They are formed by preparing a monomolecular film containing a polyamino acid and lipid on a substrate. The material to be tested is accumulated on a polymer base material. See also JP 2001/078766-A, which discloses another biochip for detecting a solution containing DNA, RNA, protein or sugar chains adhered to substrate. This chip includes an electrophoresis area and hydrolization area. In general, any suitable biosensor technology employing electrophoresis can be employed.

Sensors to monitor nitrates. For example, the Nitrate Elimination Co., Inc. (NECI) is developing an electronic device to detect nitrate using the enzyme nitrate reductase as the functional unit. Their “Nitrate Biosensor” relies on the ability of nitrate reductase to use electricity to drive the catalytic reduction of nitrate to nitrite. This concept is employed in the EzNET™ System from NECI. Small amounts of the enzyme (NaR) are coupled to an electrode providing current for the reduction of nitrate to nitrite. A digital display can report the amounts of nitrate converted. Other sensors for nitrates are disclosed in J. W. Aylott, et al. “Optical Biosensing of Nitrate Ions Using a Sol-Gel Immobilized Nitrate Reductase,” Analyst, Vol. 122, No. 1, 1997, pp. 77-80. Sensors measuring nitrite in the urine can indicate the presence of a urinary tract infection and can be usefully incorporated into diapers, bed pads, incontinence devices, menstrual pants, or other absorbent articles that can collect urine. One biosensor for detecting nitrite in urine is the test strip of Biotel Corporation (Oak Park, Ill.), which changes color if nitrites are present.

Capacitive biosensors in which changes in the dielectric properties of an electrode surface are detected. See, for example, G. Johansson, et al., “Capacitive Biosensors,”. Electroanalysis, Vol. 13, No. 3, March, 2001, pp. 173-80. In such sensors, the binding of an analyte to an immobilized affinity element can be detected directly without the need for a label or an indicating reaction. According to Johansson, et al., changes in capacitive sensors can be detected by measuring the electrical capacitance or impedance either by interdigitated electrodes or potentiostatic methods. Such biosensors have been used for detection of antigens, antibodies, proteins, DNA fragments, and heavy metal ions. Extremely low detection limits have been reported with plugged, self-assembled recognition layers.

Stochastic sensors, such as those described by H. Bayley and P. S. Cremer, “Stochastic Sensors Inspired by Biology,” Nature, Vol. 413, No. 6852, Sep. 13, 2001, pp. 226-31. They disclose use of a variety of membranebound receptors, including responsive ion channels, to discriminate between multiple stimuli. They further disclose the use of engineered membrane pores to make sensitive biosensors with potential applications that range from the detection of biological warfare agents to pharmaceutical screening. Engineered pores in this technology can detect the identity of an analyte as well as its concentration.

Disposable screen-printed sensors (SPE) optionally coupled with differential pulse voltammetry (DPV) for detection of chemical compounds, such as the sensors described in C. Capannesi et al., “Electrochemical Sensor and Biosensor for Polyphenols Detection in Olive Oils,” Food Chemistry, Vol. 71, No. 4, 2000, pp. 553-62.

A difference interferometric slab optical waveguide (SOWG) sensor can be used, such as one using a prism coupling method for flow analysis, as disclosed by K. Tsunoda, et al., “Characteristics of Sensor Response of a Difference Interferometric Slab Optical Waveguide Refractive Index Sensor with a Prism Coupling Method,” Analytical Sciences, Vol. 15, No. 3, March 1999, pp. 241-47.

The analytical products of Biosite Incorporated, including tests for drug abuse.

Miniaturized free-flow electrophoresis systems incorporating dedicated sensors for real-time analysis, such as those under development by Leatherhead in the UK.

Transdermal sampling devices coupled with sensor means and a microprocessor, such as those disclosed in WO 99/58051, which discloses a sampling system that extracts analyte from skin or mucous membranes with iontophoretic sampling to continuously measure the analyte. Transdermal sampling devices are also disclosed in EP 1,077,634, and, for glucose measurement, in WO 99/58050. U.S. Pat. No. 6,059,736 issued to Tapper and previously incorporated by reference, also discloses transdermal sampling and detection methods.

Urine sensors: GB 2348032 or U.S. Pat. No. 6,203,496, “Apparatus with Reagents for Detection of Medical Conditions,” issued Mar. 20, 2001 to Gael et al. The latter employs a color change reaction to detect an analyte in urine that can indicate the presence of a urinary tract infection, hematuria, glycosuria, biliary abnormality, ketonuria, and proteinuria.

Nanosensors using microbes to detect bacteria or tumor cells, such as the LEXAS™ and BCRS™ sensors of BCR Diagnostics (Jamestown, R.I.), and the associated technologies disclosed in U.S. Pat. No. 5,792,617, “Cell Proliferation-Based Amplified Detection of Analytes,” issued Aug. 11, 1998, and U.S. Pat. No. 5,472,846, “Test Kit and Method for Amplification and Detection of Antigen Cells,” issued Dec. 5, 1995.

The devices of Yoreh Biotech, Israel, including biosensors for cytotoxicity, and including the biosensors disclosed in WO 01/34788.

The biosensor system of JP 3127599, including electrodes, a reaction layer including hydrophilic polymer and an enzyme and an electron acceptor.

The devices of UMD, Inc., such as those disclosed in U.S. Pat. No. 6,197,327, issued Mar. 6, 2001 to Harrison et al., incorporated herein by reference, which discloses a device and method for treatment of dysmenorrhea including an intravaginal drug delivery system containing a pharmaceutical agent that can be released into the vagina and absorbed through the vaginal mucosa to provide relief of dysmenorrhea. The drug delivery system can be a tampon device, vaginal ring, pessary, tablet, suppository, vaginal sponge, bioadhesive tablet, bioadhesive microparticle, cream, lotion, foam, ointment, paste, solution or gel. The system delivers a higher concentration to the muscle of the uterus, the primary site for the dyskinetic muscle contraction, which is the pathophysiologic cause of dysmenorrhea.

Various kits for the detection of particular antibodies and/or DNA, as disclosed in WO 01/20328.

Metallized nanospheres for detecting biological analytes, using, for example, the technology of Rice University described at composite.about.com/library/PR/1999/blrice1.htm and www-ece.rice.edu/˜halas/research.html.

Disposable optical sensor chips, such as those disclosed by K. Schult et al., “Disposable Optical Sensor Chip for Medical Diagnostics: New Ways in Bioanalysis,” Anal. Chem., Vol. 71, No. 23, 1999, pp. 5430-35. The optical sensor system described therein is said to permit for all kinds of immunochemical assay formats and consists of a disposable sensor chip and an optical readout device. The chip is built up from a ground and cover plate with in- and outlet and, between, of an adhesive film with a capillary aperture of 50 μm. The ground plate serves as a solid phase for the immobilization of biocomponents. In the readout device, an evanescent field is generated at the surface of the ground plate by total internal reflection of a laser beam. This field is used for the excitation of fluorophore markers. The generated fluorescence light is detected by a simple optical setup using a photomultiplier tube. With this system, the pregnancy hormone chorionic gonadotropin (hCG) could be determined in human serum with a detection limit of 1 ng/mL.

The biosensors of U.S. Pat. No. 6,200,817, assigned to Litmus Concepts, issued Mar. 13, 2001, incorporated herein by reference, which discloses a method for detecting clue cells in vaginal fluid for diagnosis of vaginal infections.

The biosensors of U.S. Pat. No. 6,200,773 assigned to Lifescan, Inc., which discloses an analyte in a hemoglobin-containing fluid that is detected using two enzymes, a dye, a nitrite salt, and a quinone derivative.

Biosensors based on bioluminescence or chemiluminescence, such as those disclosed in U.S. Pat. No. 6,287,871, “System for Determining Analyte Concentration,” issued Sep. 11, 2001 to Herron et al., which discloses an optical detection system that detects fluorescence from fluorescent binding assays and can include a processing system to determine the analyte concentration from the detected fluorescence.

Nutritional biosensors for measuring the presence of a nutrient in the body. These include urine based tests for calcium, vitamin C, and other vitamins and minerals, saliva-based tests for zinc (e.g., the Zinc Taste Test Kit marketed by Healthy Solutions International, Hawaii, listed at www.regeneration.com/moreedk.htm#Zinc Taste Test Kit).

Biosensors may detect ammonia, urea, or other nitrogenous compounds found in body fluids. Examples of urea sensors are disclosed in A. Senillou, et al., “A Miniaturized Urea Sensor Based on the Integration of Both Ammonium Based Urea Field Effect Transistor and a Reference Field Effect Transistor in a Single Chip,” Talanta, Vol. 50, No. 1, Aug. 23, 1999, pp. 219-26, and in C. Eggenstein et al., “A Disposable Biosensor for Urea Determination in Blood Based on an Ammonium-sensitive Transducer,” Biosensors and Biotechnology, Vol. 14, 2000, pp. 33-42. Likewise, S. de Marcos, et al. in “Characterization of a Urea Optical Sensor Based on Polypyrrole,” Mikrochimica Acta, Vol. 130, No. 4, 1999, pp. 267-72 describe an optical biosensor for urea based on the enzymic reaction with urease. The enzyme was photoimmobilized with polyacrylamide on to a chemically polymerized polypyrrole film in which the polypyrrole acts as both the matrix and the indicator dye. These films so formed exhibited a change in IR absorbance in the NIR range that was pH dependent and so urea concentration dependent due to the changes in pH resulting from the urease reaction. The absorbancemeasured at 650 nm was directly proportional to the urea concentration. The sensors are said to be inexpensive and easy to prepare.

The biosensor systems of Meridian Bioscience, Inc. (Cincinnati, Ohio), which produces rapid, one-step devices for the simultaneous detection of bacteria, parasites, and other pathogens such as rotaviruses, yeast, staph bacteria, microsporidium, strep bacteria, E. coli, and the like. One exemplary product for Giardia and Cryptosporidium is ImmunoCard STAT® Cryptosporidium/Giardia. The rotavirus strips of Bio-X (Marche-en-Famenne, Belgium) for detecting viruses in feces with lateral immunochromatography can also be used.

Lifestream Technologies® Personal Cholesterol Monitor by Lifestream Technologies, Post Falls, Id.

Biosensor chips can be made from photolithographic techniques and can include, for example, one or more electrodes, such as three electrodes or more, to provide working, counter and reference electrodes of any size and shape. Sensor chips can have any suitable surface: hydrophobic or hydrophilic; acidic, basic, or neutral; high charge density or no charge; extended matrix or no matrix.

In some embodiments, healthcare can also be enhanced by monitoring and controlling the quality of the environment of the subject and of food, beverages, and other substances taken in by the patient. For example, sensors tracking pollen content, relative humidity and air temperature in a room may provide information that can be coupled with other sensor readings for a patient suffering from respiratory illness. Sensors may track drinking water quality, alerting the patient and/or caregivers when there are unacceptable agents present. Many of the operating principles for biosensors for human condition monitoring described herein can be applied to sensors for monitoring environmental conditions or food and water quality. Exemplary sensors for detecting endocrine disrupting compounds (hormone mimics) in water are described in A. M. Sesay and D. C. Cullen, “Detection of Hormone Mimics in Water Using a Miniturised SPR Sensor,” Environmental Monitoring and Assessment, Vol. 70, Nos. 102, July 2001, pp. 83-92, which describes the use of a miniature integrated surface plasmon resonance (SPR) liquid sensor from Texas Instruments. A domestic laundry detergent was used to remove immobilized assay components between each assay cycle. Water sensors can measure any suitable pollutant or other agent in the water, including arsenic, lead, chlorinated compounds, bacteria, viruses, and the like.

Additional sensors pertaining to food safety, water quality and nutrition include those investigated by the Leatherhead Food Research Association (Leatherhead, Surrey, England), disclosed at www.lfra.co.uk/candr/techinnov.htm. As discussed therein, analytes that can be detected with biosensors include the following:

Food-grade polysaccharides (e.g. carrageenans, alginates, xanthan, galactomannans, gum arabic and chitosan)

Food borne pathogens and bacterial toxins (e.g. Salmonella, Listeria, E. coli and Staph enterotoxins)

Food spoilage organisms (e.g. bacteria, yeasts, fungi)

Vitamins of the B-complex group (e.g. biotin and folic acid)

Food allergens (e.g. peanut, hazelnut, egg, soya and wheat)

Caramel colors.

The range of techniques covered includes ELISAs (based on membrane, dip-stick and microtitre plate) with appropriate end-points (e.g. colorimetric and chemiluminescent), molecular imprinting for the real-time analysis of food contaminants and components, gel-based systems (e.g. radial immunodiffusion, double immunodiffusion and immunoelectrophoresis) and real-time biosensors (e.g. Pharmacia Biacore biosensor). Sensors can also be used that employ molecular interactions (e.g. protein/protein or protein/polysaccharide) and biomodification (e.g. enzymic) of biopolymers in real time using optical sensing. Other commercially available test kits include those for food borne pathogens, antibiotics, food allergens, β-agonists and vitamins. ELISAs, for example, can be suitable for food borne pathogens and microbial toxins.

### b. Biosensors in Absorbent Articles

Methods for incorporating biosensors in absorbent articles such as diapers or sanitary napkins are disclosed in U.S. patent applications Ser. Nos. 09/299,399; 09/517,441; 09/517,481; 09/342,784; 09/342,289; and in U.S. Pat. Nos. 6,186,991 and 5,468,236, all of which have been previously incorporated by reference. Any of these can be adapted for use with the present invention.

Methods have been disclosed for providing wetness indicators or other sensors in products such as diapers. For example, U.S. Pat. No. 3,460,123 of Bass discloses a wetness detector that emits a radio signal when a diaper is wetted. Related disclosures include U.S. Pat. No. 4,106,001 of Mahoney; U.S. Pat. No. 4,796,014 of Chia; U.S. Pat. No. 5,959,535 of Remsburg, which includes sending a signal to an FM radio receiver when a diaper is wetted; U.S. Pat. No. 5,570,082 of Mahgerefteh et al.; and U.S. Pat. No. 5,838,240 of Johnson; each of which is incorporated herein by reference. Sensors for detecting odor in diapers due to defecation are disclosed by D. Yoshiteru et al., “Development of the Sensor System for Defecation,” Ishikawaken Kogyo Shikenjo Kenkyu Hokoku (Report of the Industrial Research Institute of Ishikawa, Japan), No. 49, 2000, pp. 5-10 (based on abstract).

A further example includes a sanitary napkin or panty liner containing a visual, pH-indicating strip that can detect an infection. The user or a care giver can manually translate the color signal into an entry into a personal data control means to convey the biosensor signal electronically, or the article can include electronic means to generate a signal from the detection means, such as an electronic pH indicator and wireless transmission of the measurement.

Biocatalytic means such as enzymes can be included in absorbent articles to cause a reaction with a targeted analyte that in turn leads to a measurable signal. For example, enzymes in a hydrogel, superabsorbent particles, or an emollient in a diaper can react with an analyte such as glucose or urea to cause a color change or electric signal that can be measured. In one embodiment, an indicator gel is used including oxidoreductase enzymes that produce hydrogen peroxide upon reaction with an analyte in a body fluid. The hydrogen peroxide can then oxidize a colorless compound to create a colored agent, or can bleach a dye, to visually indicate the presence of the analyte.

### c. Electronic Systems

Numerous electronic systems have been developed to monitor sensor signals, store data, transmit signals to professionals, and the like, any of which can be employed in the present invention.

The information processing in the present invention can occur on a single central server, but generally requires sharing of information across multiple servers belonging to multiple entities. A central server can be used to handle core data and its allocation to various entities in a manner that protects the privacy of the patient. Peer-to-peer (P2P) and business-to-business (B2B) approaches can be adapted for use in the present invention, as well as other models such as P2B (person-to-business).

Any suitable hardware and software can be used. Internet hubs, switches and routers, for example, or Microsoft Windows-based systems and UNIX-based can be used. Apache Web server software may be used. Server security can be provided with suitable hardware and software systems. For example, Internet firewall software by Celestix Networks can be used. Communication between servers can occur, for example, over a LAN (e.g., via an Ethernet or a Token Ring network), a wireless local area network (WLAN) using infrared (IR), ultrasonic, radiofrequency (RF), acoustic, or other wireless transmission means (including the telematic system proposed in EP 0 970 655 A1, published Jan. 12, 2000, disclosing the use of mobile phones for transmitting glucose information to a central location), a secure Intranet or via a secure Web-based system. Networks may be switched, optical, or use other technologies. Groupware systems can be employed, which use computer networking technology to allow multiple systems and individuals to communicate. The Lotus Notes/Domino system, for example, can be used to support communication between servers and Web-based applications for Intranets and other systems. Novell Groupwise is another example. The Groove system of Groove Networks, Inc. can also be used. This system includes synchronization technology that stores data for intended recipients that are offline and later forwards that data when the recipients eventually re-connect. Groove is an extensible platform and can be expanded or customized using the Groove Development Kit.

Customized applications for the present invention can be written in code from any appropriate programming language, such as C++, FORTRAN, Perl, and Python, or by using HTML web pages. Data elements can be exchanged using electronic data interchange or extensible markup language (XML). In one embodiment, a Web-based system can be used for one or more aspects of the present invention, including establishing user options and entering a privacy input, providing a display of biosensor information for the user or outside parties, for administration of data allocation and processing, for retrieval of medical records, and the like. A Web-based system can incorporate one or more databases and can employ any server such as SQL or Oracle database servers. A Web-based system also can employ XQuery, an XML query language, as described by Charles Babcock, “The Ask Master: An XML Technology Makes Retrieving Web Data Much Easier,” Interactive Week, Sep. 24, 2001, p. 48, and further described at http://www.w3.org/TR/xquery. An XQuery system, for example, could query a relational database such as a medical records database and user authentication database, as well as electronic data provided via Web pages or e-mail, incorporating data from several sources into a single XML document or Web page. The Web-based environment may be secured by any suitable means.

Many tools such as encryption are known for providing secure transmission of data. Special precautions may be desired when wireless transmission of data is used. The IEEE Wired Equivalency Protocol (WEP) can be used. To increase security, WLAN access points can be placed outside the firewall of the network or the central server, and WLAN boxes can be required to use a Virtual Private Network (VPN) to access the network. WLANs can be provided through a variety of vendors such as Catalyst International, Select, Inc., Advanced Technology Solutions (ATS), and Luna Communications. Hardware components can include, for example, Proxim Harmony Wireless units. For facilities containing a plurality of subjects with biosensors, one exemplary embodiment entails use of a Proxim Harmony 801.11b wireless network infrastructure for the facility, which can be provided through ATS. Cisco Aironet bridges can also be used for higher levels of security, due to their 128-bit encryption and Direct Sequence Spread Spectrum (DSSS) technology (see Fred Aun, “Bank on Wireless,” Smart Partner, Sep. 10, 2001, pp. 12-16). Examples of hardware for wireless access points include the modular Lucent OriNoco AS-2000 Access Point (permitting migration to future IEEE 802.11 high-speed technologies) or the AP-500 Wireless Access Point, which can be connected to a computer, for example, with an ORiNOCO PC Card.

Hardware and software systems specific to medical data and healthcare can play a role in the scope of the present invention. For example, Agilent has developed hardware and software for monitoring a patient and having results transmitted to a doctor, which can be adapted for home care or care in other settings. LifeChart.com also offers monitors for several illnesses (e.g., asthma) that involve electronic transmission of results to a doctor using secure software on the Internet. Medscape offers products that provide electronic charts that a doctor can readily update.

Parkstone Medical Information Systems offers a handheld device to permit doctors to enter notes, look up information on drugs, and place an order to the patient's pharmacy. Partners with drug companies to give preference to certain drugs, or with HMOs to offer generic drugs preferentially. Handheld devices used by doctors or patients can then be linked to a network and participate in the functions of the present invention (e.g., to receive raw data or interpreted data from the biosensor). The i-STAT® Portable Clinical Analyzer, for example, can be used in conjunction with i-STAT cartridges for the simultaneous quantitative determination of specific analytes in whole blood. Some handheld devices contain a medical dictionary and pharmaceutical tools, and may hold medical records and best-practice treatments, as described in Interactive Week, Mar. 19, 2001, pp. 26-29 (especially. p. 28).

Smart card technology can be used in the context of the present invention. Smart cards are small, portable cards that contain electronic memory (a memory chip) and may contain a microprocessor. They can be used to acquire information from a biosensor signal, to verify the identify of the user, and to provide the stored data for subsequent processing such as for analysis and display of tentative results for review by the user and transmission to an outside source. With smart cards or other dataloggers, transmission of data can also include physically transporting the smart card to a medical office or other facility, where data from the smart card can be directly downloaded into a private network or onto the computer or other data storage device controlled by an outside source. Smart cards can be customized to provide unique user information, including social security number, billing information, insurance specifications, and personal health history or various components of medical records. In one embodiment, the smart card receives both data from a biosensor signal and the privacy input from the user, and can generate a response signal, store a response signal for subsequent transmission, or provide the information required for another device to generate the response signal.

Exemplary smart cards include the Health Smart Card of Health Smart Card, Inc. (El Paso, Tex.); the Data Concern™ Smart Card of by Lifestream Technologies (Post Falls, Id.); and the proposed MoReHealth (Mobile Records for better Health). Such smart cards can contain the subject's medical history and other information in addition to store biosensor data. The data can be accessed by doctors or others with a smart card reader and additional software or hardware, as required. Researchers at the University of Newcastle, Australia, have proposed the MoReHealth smart card containing medical records with privacy protection, in which doctors could access more information than pharmacists, who would only be able to access prescription details and not to medical records, according to the article, “Smart Card Makes Medical History,” ZDNet Australia, 18 Jul. 2001, available at www.zdnet.com.au/newstech/enterprise/story/0,2000025001,20243364-1,00.htm.

Smart cards for use in the present invention can require physical contact for reading or transmitting a signal or can be contactless (i.e., capable of reading a signal or providing a signal via an electronic impulse sent through the air from or to an antennae or similar device). Smart cards can store a PIN to improve security, but they can also add biometric identifiers: voiceprints, fingerprints, retina scans, iris scans or dynamic signature patterns. For example, Sense Holdings Inc. (Tamarac, Fla.), through its Sense Technologies subsidiary, has developed a fingerprint-based smart card (the BioCard™ system) to provide enhanced security for storing and accessing portable data, including medical care information. An example of a card with a thin battery is disclosed in U.S. Pat. No. 6,284,406, “IC Card with Thin Battery,” issued Sep. 4, 2001 to Xing et al., incorporated herein by reference. In one embodiment, the smart card includes display panel such as a liquid crystal, LED, or liquid paper display panel for displaying a graphical portrayal of the biosensor signal or data derived therefrom. In yet another embodiment, the smart card includes input means or is attached to input means for receiving a privacy input from the user. Input means can include a button (physical or graphics based on a display panel) or other sensor for indicating a yes or no answer, or can include other means for receiving textual input as well or for selection of a predetermined input from a variety of predetermined input choices using a menu, button, or other selection means.

Some people have expressed concern about the use of smart cards and the possibility of loss of privacy. Thus, in one embodiment of the present invention, smart cards are not used.

Medical “telesensors” are described at www.ornl.gov/ORNLReview/rev29_{—}3/text/biosens.htm. In one example, a minute chip measures body temperature or pulse and transmits a signal to a local receiving and transmitting device, such as a device in the helmet of a soldier. The receiving and transmitting device can then send a signal to a distant station to call of emergency help or to allow tracking of the location of the wearer.

Biosensors in absorbent articles are disclosed in the following P&G patent applications, with some teachings related to wireless signal transmission: WO 00/65347; WO 00/65348; WO 00/65084; and WO 00/65096.

Regarding the storage of electronic medical records, any suitable methods can be used, including the systems disclosed by Kameda and ltoh in U.S. Pat. No. 5923018, “Medical Care Schedule and Record Aiding System, Medical Care Schedule and Record Aiding Method, and Program Storage Device Readable by the System,” issued Jul. 13, 1999, incorporated herein by reference.

### d. Drug Delivery

Biosensors can work in tandem with drug delivery systems. For example, a disposable article or article of clothing may carry or support a biosensor cooperatively associated with a drug delivery system, such that the manner of administrating the drug (including the dose delivered or the frequency of application) or the manner of delivering any therapeutic treatment is influenced by the biosensor. Also by way of example, a drug delivery device may be responsive to an electronic signal generated by a biosensor or in response to a biosensor reading. When the biosensor indicates that an analyte is above or below a predetermined range, then the drug delivery device may be activated or modified to change the manner of application of a drug in response to the condition indicated by the level of the analyte detected.

The drug delivery device can be automatically activated or modified, or may require manual activity to affect the change. Manual activity can involve the wearer or a nurse or other party adjusting a setting on a drug delivery device in response to a biosensor reading, or can involve human verification of an automatic change proposed by a drug delivery system in response to a signal from a biosensor.

One system illustrating a means of employing a biosensor to influence drug delivery for a patient is disclosed in U.S. Pat. No. 6,059,736, “Sensor Controlled Analysis and Therapeutic Delivery System,” issued May 9, 2000 to R. Tapper, previously incorporated by reference. Electro-osmotic means coupled with a biosensor are used to sense various analytes withdrawn noninvasively from the body of the subject, followed by delivery of a drug at a dosage responsive to the reading from the biosensor. The work of Tapper can be applied to glucose measurement and control, but may also be applied to measure other substances such as urea, creatinine, lactate, cholesterol, aspirin and paracetamol (a pain relief substance). Further, in the work of Tapper, a D.C. signal is used to obtain elevated drug delivery levels, allegedly without skin injury or pain.

Another example of osmotic drug delivery means, which can be used in the scope of the present invention, is disclosed in U.S. Pat. No. 6,283,953, “Osmotic Drug Delivery Monitoring System and Method,” issued Sep. 4, 2001 to Ayer et al., incorporated herein by reference. Means for noninvasively measuring the release of the drug from an implanted device are disclosed by Ayer et al.

A drug delivery device for use with the present invention can also be physically attached to or integrated with the biosensor. For example, the biosensor and/or treatment means for the subject can include the silicon needles of Yuzhakov et al. disclosed in WO 00/74765, “Intracutaneous Microneedle Array Apparatus,” published Dec. 14, 2000, claiming priority to U.S. patent application Ser. No. 09/239,025, filed Jun. 9, 1999, incorporated herein by reference. The biosensors and/or treatment means of the present invention can also include the microneedle devices of Prausnitz et al. disclosed in WO 00/74763, “Devices and Methods for Enhanced Microneedle Penetration of Biological Barriers,” published Dec. 14, 2000, which claims priority to the following U.S. patent applications, each of which is incorporated herein by reference: U.S. Ser. Nos. 60/137,621, filed Jun. 4, 1999; 60/146,200, filed Jul. 29, 1999; 09/448,107, filed Nov. 23, 1999; 09/452,979, filed Dec. 2, 1999; and 09/453,109, filed Dec. 2, 1999. In the application of these systems to the present invention, one or more silicon microneedles penetrating the stratum corneum are adapted to measure a biological condition in the blood or tissues of the body, such as the presence of an analyte, a pH value, a conductivity value, response to an electrical discharge, a signal detected from a thin fiber-optic probe integrated with a silicone needle, and the like. Based on what is detected by a probe or other sensor associated with one or more silicon needles or with the silicon needle patch itself, other silicon needles in the device may delivery a therapeutic treatment. Hollow microneedles, for example, may release a pharmaceutical agent or other compound into the skin for intake by the body and/or localized delivery. Needles may also receive an electrical signal to cause release of metal ions into the skin, such as copper ions or zinc ions, released electrochemically. A voltage may also be applied into the skin to activate a compound or further increase transdermal diffusivity for enhanced delivery of a topically applied agent, which can be applied before or after application of a microneedle patch to the skin, or may be applied to the skin while the patch is in place, such as by release from small ports or pores in a portion of the patch.

Rather than release or application of a drug in response to a biosensor signal, a secondary agent may be applied which improves transdermal delivery of a drug that may already be present, or that increases the biological uptake or effectiveness of a drug that may already be present.

U.S. Pat. No. 6,274,166, “Transdermal Delivery System,” issued Aug. 14, 2001 to A. Sintov et al., discloses the use of permanganate or silver protein to increase transdermal drug delivery for insulin or diabetes. Such compounds or other agents suitable to enhance transdermal drug delivery can be administered in response to a biosensor signal, followed by the optional application or increased application of the drug itself.

Drugs may be present in a disposable article, such as a medicated and instrumented tampon or sanitary napkin. Drug delivery may depend upon the presence of moisture to permit release of the drug, as in time-release capsules made of gelatin or other water soluble materials, including “XGel” materials, which are gels and films made from polyvinylalcohol or a cellulose derivative that can replace gelatin capsules used for medication, as described more fully in Materials World, June 2001, pp. 10-12. The delivery of the drug may be substantially independent of the biosensor signal, or can be regulated or modified in response to the signal.

Active ingredients such as agents for comfort of the skin, pharmaceutical compositions, and the like, etc., can be encapsulated in slow-release material, including starch-polyvinyl alcohol matrices as described, for example, in Z. Zhu and R. Zhuo, “Slow Release Behavior of Starch-g-poly(vinyl alcohol) Matrix for 2,4,5-Trichlorophenoxyacetic ACOD Herbicide,” European Polymer Journal, Vol. 37, No. 9, September 2001 (published Jul. 6, 2001), pp. 1913-19; or polysaccharide-borate complexes such as those described in B. S. Shasha et al., “Starch-borate Complexes for EPTC Encapsulation,” J. Appl. Polymer Science, Vol. 29, pp. 67-73 (1984). Other chemistries for encapsulation with starch compounds include those disclosed in R. E. Wing and B. S. Shasha, “Encapsulation of Organic Chemicals within a Starch Matrix,” J. Chem. Educ., Vol. 60, 1983, pp. 247-48; B. S. Shasha et al., “Encapsulation of Pesticides in a Starch-calcium Adduct,” J. Polymer Science: Poly. Chem. Educ., Vol. 19, 1981, pp. 1891-99; and E. I. Stout, et al., “Pilot Plant Process for Starch-xanthide Encapsulated Pesticides,” J. Appl. Polymer Science, Vol. 24, 1979, pp. 153-59.

### e. Other Embodiments

Financial aspects of biosensor use can also be considered. There may be a charge for each data set the patient transmits for consideration by a doctor. The patient may also be charged daily for using a biosensor that provides regular or continuous information to a central database or to health care personnel for monitoring. Higher costs may be charged for the privilege of maintaining control over data.

Biosensors in the present invention may be further augmented with transmitters that can be used to identify the location of the subject, such as a personal GPS system or radio signal emitter. In one embodiment, the health status of a person can be monitored along with physical position, which may be especially useful for children, explorers or hikers, soldiers, and the like.

### f. EXAMPLE 1

A child in a day care institution is monitored using a plurality of biosensors contained in disposable and durable clothing. The disposable article could be a diaper or HUGGIES® Pull Ups® instrumented with multifunctional sensors for detecting the presence of moisture (e.g., according to U.S. Pat. No. 6,200,250, incorporated herein by reference, which disclosed electrodes in a diaper for sensing moisture, or any other suitable method) and one or more analytes in urine. Sensors in a shirt could measure body temperature, heart rate, and one or more analytes obtainable through the skin such as osmotically obtained glucose or cortisol. The biosensor signals could be transmitted by radiofrequency to a local receiver connected to the Internet, permitting a parent to access a secure Web page where real-time and historical biosensor data for the child could be viewed.

The privacy input in this case is a previously determined setting entered by the parent, the representative of the child, which indicates who can access all or portions of the data that is sent to an Internet source, and what signals may be sent to whom depending upon the nature of the biosensor signal. Access to the data can be achieved by logging in with a user ID and password that determines what can be accessed by the person logging on. Based on the privacy input, a portion of the biosensor signals may be made available to a pediatrician or may be archived. The privacy input can call for automatic contacting of outside parties such as one or both parents, another relative, or emergency personnel if the biosensor signal indicates a life-threatening situation or other condition calling for a response by a caregiver or other party.

Biosensor data possibly indicative of an infectious disease may be used, in accordance with the privacy input, to alert the day care staff so that the risk of the infection spreading to other children may be reduced. The day care institution, in some cases, may require by contract that such data be provided to assist them in protecting the health of other children.

The biosensor signal may also be coupled with additional electronic signals, such as an audio signal from a miniature microphone in the child's clothing that permits parents to listen to the setting in which the child is located. One or more video signals from videocameras in the day care center may also be available and provided via Internet, possibly as a service of the day care institution. In one embodiment, a secure Web page for the child allows the parent to see and listen to the day care environment while monitoring physiological data for the child.

Similar systems could be adapted for remote monitoring and responsive caregiving (or other responsive steps) for the wellbeing of any person in any setting, such as a prison inmate, a student, a person in a nursing home or hospital, and the like.

### g. EXAMPLE 2

A prophetic example is suggested for the management and regulatory compliance of a herd of dairy cows using biosensors. As has been disclosed in the article “Biosensors to Detect Oestrus,” Silsoe Research Institute News, Issue 6, Autumn 1999, available online at www.sri.bbsrc.ac.uk/news/Autumn99/Biosensors.htm, enzyme linked immunoassay (ELISA) can be applied to milk samples to map the ovulation cycle for cows. Formerly, tests required manual sampling and analysis by the farmer, but SRI has proposed adapting biosensor techniques to emulate the ELISA tests automatically in the milking system and is developing automated ovulation prediction systems for dairy cows. They propose integrating a biosensor with automatic milk sampling and a herd management database. The proposed biosensor is a screen printed carbon electrode system. Such a milk monitoring system could be further expanded to monitor bovine growth hormone, white blood cells due to mammitis, vitamins, calcium content, fat content, and other nutritional and safety factors with online biosensors in contact with milk being withdrawn from cows, as well as from biosensors in cooperative association with the body of a cow. The herd management database belonging to the farmer could further be networked with outside sources, to which information from the milk and mammal biosensors could be made available via a data allocation and processing module, as regulated by the farmer who could enter a privacy input. Restriction of data access to outside agencies, beyond that established by a predetermined default setting, such as wholesale organizations or regulatory agencies, would normally require justification and could result in loss of sales or suspension of a license. But even when data were not restricted, the privacy input could provide a valuable means to annotate the results or explain problems with equipment, or steps taken to manage an epidemic or other problems to satisfy regulatory or quality control burdens.

In one embodiment, the invention provides a healthcare network for sharing information concerning the health of a user with one or more outside sources, the network including a biosensor cooperatively associated with the user that generates a biosensor signal pertaining to the health of the user; and a personal data control means including means for receiving the biosensor signal, input means for receiving a privacy input from the user or representative of the user, and output means for generating a response signal based on the biosensor signal and privacy input. The network also includes a data allocation and processing module including means for receiving the response signal from the personal data control means and means for directing one or more output signals to the one or more outside sources, responsive to the response signal, wherein the availability to the one or more outside sources of health-related information pertaining to the user is responsive to the privacy input.

The outside source may be a physician, a hospital employee, an employer, a pharmacist, a nurse, a public officer, or a provider of services or materials intended for the well-being of the user. The network may also include data storage means to archive health-related information pertaining to the user in the form of electronic medical records. The network may also include treatment means for delivering a medication, nutritional substance, medical therapy, or other physical or medical care to the user responsive to the output signal to the one or more outside sources.

The biosensor may be provided in a disposable article worn that contacts at least one body fluid from the user. The biosensor may measure one or more analytes in blood. The biosensor may also measure one or more analytes in at least one of menses, feces, or urine. The biosensor may also measure one or more analytes in at least one of nasal secretions, sweat, or saliva. The biosensor may also measure one or more analytes taken from an invasively withdrawn biological sample. The biosensor may noninvasively measure one or more analytes from the body of the user. The biosensor may measure an analyte in a gaseous medium. Finally, the biosensor may employ antibodies for detection of biological analytes.

The data allocation and processing module and at least one element of the personal data control means may include a common electronic data processing device. The biosensor may noninvasively detect glucose in the blood. Insulin may be delivered to the body of the user responsive to the biosensor signal.

The personal data control means may include a data acquisition device, a visual display related to the biosensor signal, and text input means for entering annotations. At least one of the personal data control means and the data allocation and processing module may include a Web-based interface. The personal data control means may include an interactive electronic display that portrays data derived from the biosensor signal and provides options for a privacy input. The interactive electronic display may include a Web-based interface adapted for secure transmission of data to the data allocation and processing module.

The network may include alert means to send an alert signal to a caregiver or representative of the user when a parameter derived from the biosensor signal falls within one or more predetermined ranges.

In an alternate embodiment, the invention provides a method for sharing information concerning the health of a user with one or more outside sources, the method including providing a biosensor cooperatively associated with the body of a user, wherein the biosensor generates a biosensor signal pertaining to the health of the user; providing a reading to the user or a representative of the user indicating a preliminary interpretation of the biosensor signal; and receiving a privacy input from the user or a representative of the user through input means. The method also includes generating a response signal based on the biosensor signal and the privacy input; and receiving the response signal at a data allocation and processing module, which in turn generates one or more output signals to the one or more outside sources, responsive to the response signal, wherein the availability to the one or more outside sources of health-related information pertaining to the user is responsive to the privacy input.

The method may also include providing an adjustment in care to the user in response to the output signal as directed by at least one of the one or more outside sources. The biosensor may measure an analyte associated with renal disease in a body fluid, and wherein the output signal includes information pertaining to renal health for review by a physician.

The present invention relates to an integrated health care system employing biosensors capable of generating signals relating to the health of the user that can be processed and transmitted as needed to various destinations, wherein the user or representative of the user maintains a degree of control over the data transmitted for protection of the user's privacy or other considerations. The invention further relates to particular combinations of sensor technologies and information management systems and/or health management systems for the benefit of the user, including embodiments wherein a degree of personal control over data sharing is maintained for user privacy.

In one embodiment, the present invention relates to a healthcare network for sharing information concerning the health of a user with one or more outside sources, including:

a) a biosensor cooperatively associated with the user that generates a biosensor signal pertaining to the health of the user;

b) a personal data control means including means for receiving the biosensor signal, input means for receiving a privacy input from the user or representative of the user, and output means for generating a response signal based on the biosensor signal and privacy input; and

c) a data allocation and processing module including means for receiving the response signal from the personal data control means and means for directing one or more output signals to the one or more outside sources, responsive to the response signal, wherein the availability to the one or more outside sources of health-related information pertaining to the user is responsive to the privacy input.

The healthcare network can further include treatment means for delivering a medication, nutritional substance, medical therapy, or other physical or medical care to the user, responsive to the output signal to the one or more outside sources.

In another aspect, the present invention relates to a method for sharing information concerning the health of a user with one or more outside sources, including:

a) providing a biosensor cooperatively associated with the body of a user, wherein the biosensor generates a biosensor signal pertaining to the health of the user;

b) providing a reading to the user or a representative of the user indicating a preliminary interpretation of the biosensor signal;

c) receiving a privacy input from the user or a representative of the user through input means;

d) generating a response signal based on the biosensor signal and the privacy input;

e) receiving the response signal at a data allocation and processing module, which in turn generates one or more output signals to the one or more outside sources, responsive to the response signal, wherein the availability to the one or more outside sources of health-related information pertaining to the user is responsive to the privacy input.

An electronic personal data control means can be used in performing steps b, c, and d in the above method. The method can further include providing an adjustment in care to the user in response to the output signal as directed by at least one of the one or more outside sources.

In one embodiment the user is monitored with at least one biosensor while at a remote location relative to a hospital or other medical care facility. For example, the user can be at home, in a managed care facility, at the user's workplace, outdoors, traveling, and the like.

A biosensor signal or a signal derived from a biosensor signal can be transmitted to a private database or databases for review by outside sources such as a physician or nurse, but the transmission of data and optionally the availability of that data to other parties is controlled by the user or representative of the user, such as a parent, family member, someone with power of attorney, or other authorized party. The user is generally human but can be another species, such as a pet or farm animal, in which case a human representative (the owner, for example) would typically provide the privacy input.

Typically, the biosensor signal is used to generate an intermediate reading or other signal that can be interpreted by a user or other caregiver, which can permit the user or representative of the user to decide whether the data or information derived therefrom should be forwarded to or made available to outside sources. Decisions about control and availability of the data can be made and revised repeatedly or can be made only once, if desired.

Means can also be provided to generate an alert signal to the user, a caregiver, or other party based on abnormal biosensor readings that may indicate an health problem. The alert signal may also automatically initiate a call to emergency personnel or application of a responsive treatment, or may require review of an outside party such as a doctor before the treatment is automatically administered. Software and hardware means may also be provided to distinguish an abnormal reading from a hardware problem, such as a disconnected electrode or improper use of the biosensor. Neural networks and fuzzy logic systems may be incorporated to make this distinction.

Private control of the data generated by a biosensor is achieved via a personal data control means, which can include hardware and software for display and tentative interpretation of the biosensor signal(s), input means for receiving a privacy input from the user or user's representative, and transmission means to direct the resulting response signal (a signal based on the biosensor signal and a privacy input from the user) to a device for data allocation and processing, where data control instructions responsive to the privacy input are used to direct one or more output signals to one or more outside parties such as a doctor, insurer, employer, and the like.

The privacy input can include instructions about how data or other information pertaining to or derived from the biosensor signal may or may not be used and with whom the data or subsets of the data may be shared. Alternatively or in addition, the privacy input can include optional comments and other restrictions pertaining to the data. In one embodiment, the privacy input can be determined by user options that the user selects prior to measurement, or can include privacy settings entered by the user after reviewing data derived from the biosensor signal.

Means may be provided to automatically override a privacy setting when the biosensor may indicate a life-threatening condition or other condition requiring emergency response, or such means may be part of an initial setting approved by the user that can override subsequent selections.

The input means for entering a privacy input can include any suitable data entry means, such as a keyboard connected to a computer, a voice recognition device, a hardware setting such as a button or dial, a toggle switch, and the like, and can be provided by software settings, as in a file specifying user options. Symbolic entry using penstrokes or other interpretable motions can also be used.

Data allocation and processing can be performed with hardware and/or software that is part of the personal data control means, or can occur on a separate server or other means. The output signal forwarded by the data allocation and processing function may then be used by professional staff or other competent parties to adjust medications or other primary care functions provided to the user, to recommend that the user be given further testing or examination, to call for emergency assistance, to authorize payment by an insurer or other party, to verify other claims made by the user, or for other purposes typically related to the well-being of the user.

A plurality of users at one or more locations may be monitored with the healthcare network of the present invention, each being monitored by one or more biosensors and each optionally having some degree of control over the use of data generated by or derived from biosensors or associated equipment.

The “outside sources” in the healthcare network can include any of the following: doctors, nurses, dentists, and other medical staff at a hospital or other care facility, medical and dental insurers, life insurance agencies, pharmacists and any other providers of medications or health care devices or therapies, public officers such as police or probation officers, employers and associated personnel (e.g., airline supervisors monitoring a pilot or military staff monitoring biosensor signals from soldiers), and so forth. Doctors can include family doctors, pediatricians, surgeons, nephrologists, hematologists, oncologists, gynecologists, dermatologists, and specialists in any other branch of medicine. The associated databases or information management systems for each of the above-mentioned entities can also be included in the healthcare network.

Turning now to the generation of the biosensor signal(s), one or more biosensors measures one or more analytes related to the health of a user (in many cases, a patient). The medium that may contain the targeted analyte can be withdrawn or collected from the user's body, such as an analyte in a body fluid or biological sample, or can be in a material to be ingested or taken in by the body of the user, such as in drinking water, a food to be consumed, or a medication to be applied (e.g., orally or intravenously). An analyte from the user's body can be obtained by collection of a body fluid or biological sample that is invasively withdrawn (e.g., blood or spinal fluid) or collected after passing outside the body of the user. The analyte need not be removed from the body of the user, as in cases where a measurement is made on or through the skin or other tissues of the body, such as optical measurement of a substance in the blood. In one embodiment, the analyte can be noninvasively withdrawn through unbroken skin or mucosal membranes by noninvasive electro-osmotic withdrawal, as disclosed in U.S. Pat. No. 6,059,736, “Sensor Controlled Analysis and Therapeutic Delivery System,” issued May 9, 2000 to R. Tapper, incorporated herein by reference. They can also be used to momentarily or continuously contact a body fluid or body fluid source.

A biosensor can be in contact with the body or in fluid communication with the body. It can be placed on or adjacent to the skin or other member of the body (generally in fluid communication therewith), in an orifice of the body, inside the body (e.g., a surgically implanted device or a device that is swallowed or introduced by a catheter), in an article that is worn next to the body, and so forth. Biosensors or components thereof can be attached to the skin with hydrogels, including poly(2-hydroxyethyl methacrylate) (PHEMA), whose methods of preparation are described, for example, in A. C. Duncan et al., “Preparation and characterization of a poly(2-hydroxyethyl methacrylate),” European Polymer Journal, Vol. 37, No. 9, September 2001 (published Jul. 6, 2001), pp. 1821-26.

Biosensors can be spaced apart from the body, such as a biosensor measuring compounds in human breath (e.g., an electronic nose) or other body odors, where they can be in vapor communication with the body. Biosensors spaced apart from the body also include those measuring material removed from the body for separate analysis, such as a blood sensor measuring analytes in withdrawn human blood. Such biosensors can be at any distance from the body, while odor sensors and the like generally should be within a predetermined distance from the body of the user (the subject) such as within 15 inches of the body or within 6 inches or 3 inches of the body (i.e., within 6 inches or 3 inches of the closest source of the analyte being measured). In one embodiment, the biosensor (particularly the sensing element thereof) is at least 1 inch away from the body, more specifically at least 3 inches away from the body.

Biosensors can be placed in disposable absorbent articles such as diapers, disposable training pants such as HUGGIES® Pull-Ups®, bed pads, sanitary napkins, panty liners, tampons, interlabial devices, colostomy bags, breast pads, incontinence devices such as incontinence pads, briefs or undergarments. They can also be placed in other devices for collection or disposal of body fluids and other biological waste matter, as exemplified by the flexible waste bags described in WO 00/65348, which can be flexible receptacles for the containment of excreted fecal matter or urine, and in waste receptacles for diapers or other disposable materials, bedpans, toilet bowls, vomit bags, and the like. Biosensors can be associated with an article of clothing such as a shirt, underwear, a vest, a protective suit, an apron or bib, a hat, socks, gloves, or a disposable gown (particularly for medical or surgical use, or for use by a patient), or can be associated with any other object that can be in contact with or near the body, such as a pillow, bed linens, a mattress, breathing tubes, a helmet, face masks, goggles, article of jewelry such as a bracelet or necklace, an ankle bracelet such as those used for prisoners or those on probation, and the like. They can also be physically associated with a wide variety of other objects, such as suppositories, tongue depressors, cotton swabs, cloth towels or paper towels, spill cleanup bags, desiccant bags, disposable mops, bandages, wipes, therapeutic wraps, supports, disposable heating pads, articles of furniture, food containers, and the like.

In specifying where a biosensor is placed, it is understood that not all of the biosensor assembly must be so placed, but that a sensing component thereof is placed in the described location to facilitate measurement. Thus, a sensing element may be placed in a diaper, while other components of the biosensor, such as a power supply or calibration element, may be located elsewhere.

Sampling of body fluids for biosensor detection can be achieved, when needed, by use of the absorbent articles described above. Blood samples and other biological samples can be obtained by any suitable means. Further, for collection of fluids such as saliva, articles with which a saliva sample can be taken, such as a tooth brush, lip stick, lip balm, toothpick, disposable wipe such as a cloth or nonwoven material, and the like can be used.

The biosensor may be in the form of dedicated hardware for repeat uses, or can be an inexpensive, disposable probe for single use or a small number of repeat uses. The biosensor can be incorporated into an article of clothing or disposable article, and can include any of the biosensor technologies and configurations disclosed in the following U.S. patent applications: Ser. No. 09/299,399, filed Apr. 26, 1999; Ser. No. 09/517,441, filed Mar. 2, 2000; Ser. No. 09/517,481, each of which are incorporated herein by reference, the contents of which are believed to have been published at least in part in WO 00/65347, published Nov. 2, 2000 by Hammons et al.; WO 00/65348, published Nov. 2, 2000 by Roe et al.; and WO 00/65083, WO 00/65084; and WO 00/65096, each published Nov. 2, 2000 by Capri et al. The biosensor can also include any of the technologies disclosed in U.S. Pat. No. 6,186,991, issued Feb. 13, 2001 to Roe et al., incorporated herein by reference, and in the U.S. applications Ser. No. 09/342,784 and U.S. Ser. No. 09/342,289, both filed Jun. 29, 1999 in the name of Roe et al., both of which are incorporated herein by reference, and both of which are related to the disclosure published as WO 01/00117 on Jan. 4, 2001. The biosensor can also be any of those disclosed in U.S. Pat. No. 5,468,236, issued to D. Everhart, E. Deibler, and J. Taylor, incorporated herein by reference. Additional biosensor technologies and systems are set forth hereafter in this document.

Biosensor signals may be continuous or discrete, and may be taken over a short period of time such as a single measurement from one biological sample, multiple measurements over a period of hours or days, continuous measurement during a prolonged period of time such as a year, and the like. Details for the analysis and use of the signals so generated in the context of a healthcare network are set forth hereafter.

More specifically, the invention provides a healthcare network for sharing information concerning the health of a user with at least one outside source, the network including a biosensor associated with the user that generates a biosensor signal containing the information; and a personal data control means including receiving means for receiving the biosensor signal, input means for receiving a privacy input from the user, and output means for generating a response signal based on the biosensor signal and privacy input. The network also includes a data allocation and processing module including means for receiving the response signal, and means for generating and directing an output signal to the at least one outside source, wherein the module is responsive to the response signal, and wherein the availability of the information to the at least one outside source is responsive to the privacy input.

While the invention has been described in conjunction with several specific embodiments, it is to be understood that many alternatives, modifications and variations will be apparent to those skilled in the art in light of the foregoing description.

Accordingly, this invention is intended to embrace all such alternatives, modifications and variations that fall within the spirit and scope of the appended claims.

What is claimed is:

1. A healthcare network for sharing information concerning the health of a user with at least one outside source, the network comprising:
a biosensor associated with the user that generates a biosensor signal containing information regarding a health condition of the user;
a personal data control means including
receiving means for receiving the biosensor signal,
input means for receiving a privacy input from the user, and
output means for generating a response signal based on the biosensor signal and privacy input; and
a data allocation and processing module including
means for receiving the response signal, and
means for generating and directing an output signal to the at least one outside source, wherein the module is responsive to the response signal, and wherein the availability of the information to the at least one outside source is responsive to the privacy input.

2. The network as in claim 1, wherein said biosensor is configured to be placed on or against the body of the user.

3. The network as in claim 2, wherein said biosensor is placeable in an article worn by the user.

4. The network as in claim 3, wherein said biosensor is placeable in an absorbent article worn by the user.

5. The network as in claim 4, wherein said absorbent article is one of a diaper, training pant, bed pad, sanitary napkin, panty liner, tampon, interlabial device, colostomy bag, breast pad, incontinence pad, brief, and undergarment.

6. The network as in claim 2, wherein said biosensor detects an analyte in a biological sample or medium from the user's body.

7. The network as in claim 2, wherein said biosensor is ingestable by the user.

8. The network as in claim 1, wherein said biosensor is spaced from the user and detects an analyte in a medium withdrawn from the user's body.

9. The network as in claim 8, wherein said biosensor is placeable in a device for collection of bodily wastes or fluids.

10. The network as in claim 1, wherein said biosensor comprises a sensing element placeable at a first location to detect an analyte in a medium from the user's body, and additional components at a second location to generate and transmit the biosensor signal.

11. The network as in claim 1, wherein said biosensor detects at least one analyte in a medium from the user, the analyte indicative of a health condition of the user.

12. The network as in claim 1, further comprising treatment means for delivering medical care to the user in response to said output signal received by said outside source.

13. The network as in claim 12, wherein said treatment means comprises means for delivering one of a medication and nutritional substance to the user.

14. The network as in claim 1, further comprising means for generating an alert signal in response to an abnormal biosensor signal.

15. The network as in claim 14, wherein said alert signal generating means transmits said alert signal to the user and emergency response personnel.

16. The network as in claim 1, wherein said personal data control means further comprises means for generating an initial interpretation of the biosensor signal for evaluation by the user.

17. The network as in claim 1, further comprising means for overriding said privacy input means in response to a biosensor signal indicative of a health condition requiring immediate attention by a medical care giver.

18. The network as in claim 1, wherein said privacy input means comprises at least one of a keyboard connected to a computer, a voice recognition device, and a hardware device.

19. The network as in claim 18, wherein said privacy input means further comprises software containing the user's privacy settings and options.

20. The network as in claim 1, wherein said data allocation and processing module utilizes hardware and software that is also a part of said personal data and control means.

21. The network as in claim 1, wherein said data allocation and processing module utilizes hardware and software operably remote from said personal data and control means.

22. The network as in claim 1, further comprising a plurality of said biosensors configured for simultaneously monitoring a plurality of users, each biosensor generating a respective biosensor signal and associated with a respective said personal data and control means.

23. A method for sharing information concerning the health of a user with an outside source, the method comprising:
providing a biosensor operatively associated with a user, wherein the biosensor generates a biosensor signal pertaining to the health of the user;
receiving a privacy input from the user through input means;
generating a response signal based on the biosensor signal and the privacy input;
receiving and processing the response signal, and generating and directing an output signal to the outside source in response to the response signal; and
wherein the availability of information contained in the biosensor signal to the outside source is controlled by the privacy input from the user.

24. The method as in claim 23, further comprising providing a reading to the user indicating a preliminary interpretation of the biosensor signal prior to the user entering the privacy input.

25. The method as in claim 23, further comprising delivering medical care to the user in response to the output signal received by the outside source.

26. The method as in claim 25, wherein the medical care includes any combination of adjusting or administering medication, adjusting or administering primary care functions provided to the user, ordering testing or examination based upon the output signal, initiating emergency response and treatment, and authorizing billing or payment related to medical care.

27. The method as in claim 25, further comprising transmitting the biosensor signal directly to emergency medical personnel in the event the biosensor signal indicates a health condition requiring immediate attention.

28. The method as in claim 27, further comprising overriding the user's privacy input to transmit the biosensor signal to the emergency medical personnel.

29. The method as in claim 23, further comprising generating an alert signal to the user in the event of an abnormal biosensor signal.

30. The method as in claim 29, further comprising transmitting the alert signal to the outside source.

31. The method as in claim 23, wherein by way of the privacy input, the user controls and selects particular outside sources to receive the output signal.

32. The method as in claim 23, wherein by way of the privacy input, the user restricts an outside source's ability to transmit the output signal or information contained in or derived therefrom to third parties.

33. The method as in claim 23, wherein by way of the privacy input, the user dictates restrictions on the outside source's internal use of the output signal and information in or derived therefrom.

34. The method as in claim 23, further comprising providing a reading to the user indicating a preliminary interpretation of the biosensor signal prior to the user entering the privacy input, the user selecting privacy input options based on the preliminary interpretation.

35. The method as in claim 23, wherein the outside source comprises a network of health care providers, the output signal being transmitted within the network of health care providers as allowed by the privacy input.

36. The method as in claim 23, wherein the outside source comprises a medical networking infrastructure to which medical hardware devices are in communication, the output signal being transmitted directly to such medical devices as allowed by the privacy input.

37. The method as in claim 23, wherein the biosensor detects an analyte related to a health condition in a biological sample or medium from the user.

38. The method as in claim 37, wherein the medium is withdraw or collected from the user's body prior to detection of the analyte by the biosensor.

39. The method as in claim 38, wherein the medium is invasively withdrawn from the user.

40. The method as in claim 37, wherein the biosensor detects the analyte in the body of the user.

41. The method as in claim 40, wherein the biosensor is placed on or adjacent to the user's body.

42. The method as in claim 40, wherein the biosensor is implanted in the user's body.

43. The method as in claim 23, wherein the biosensor is placed in an article worn by the user.

44. The method as in claim 43, wherein the biosensor is placed in an absorbent article worn by the user.

45. The method as in claim 23, wherein the biosensor is placed in a collection device for bodily fluids or waste.

46. The method as in claim 23, comprising monitoring for a health condition with the biosensor on a generally continuous basis.

47. The method as in claim 23, comprising monitoring for a health condition with the biosensor on an intermittent basis.

48. The method as in claim 23, wherein the biosensor is a single use disposable item.

49. The method as in claim 23, wherein the biosensor signal provides a qualitative measurement.

50. The method as in claim 23, wherein the biosensor signal provides a quantitative measurement.

51. The method as in claim 23, further comprising providing the user with confirmation that the output signal has been transmitted to and received by the outside source.

52. The method as in claim 51, further comprising notifying the user of when and by whom the output signal has been received.

53. The method as in claim 23, further comprising electronically notifying the user of a health care provider's planned course of action in response to the output signal prior to implementing such a course of action.

54. The method as in claim 23, wherein the biosensor signal is a time-averaged signal derived from a plurality of measurements taken over a period of time.
